(19) United States
(12) Patent Application Publication
(10) Pub. No.: US 2009/0136553 A1
(43) Pub. Date: May 28, 2009

(54) TRIGGERABLY DISSOLVABLE HOLLOW FIBERS FOR CONTROLLED DELIVERY

(76) Inventors: Jorg C. Gerlach, Pittsburg, PA (US); Jianjun Guan, Dublin, OH (US); Steven R. Little, Allison Park, PA (US); Alan J. Russell, Gibsonia, PA (US); Jillian Tengood, Pittsburgh, PA (US); William R. Wagner, Wexford, PA (US)

Correspondence Address: JESSE A. HIRSHMAN, ESQ., 1722 MURRAY AVENUE, THIRD FLOOR, PITTSBURGH, PA, 15217, US

(21) Appl. No.: 12/237,835

(22) Filed: Sep. 25, 2008

Related U.S. Application Data
(60) Provisional application No. 60/974,922, filed on Sep. 25, 2007.

(51) Int. Cl. A61F 2/04; C07H 3/00; C08G 63/44; A61K 38/18; C12M 3/00; C08L 89/00; C12N 5/06

(52) U.S. Cl. 424/422; 536/123.1; 536/69; 528/363; 525/54.1; 435/400; 514/12; 435/325; 435/293.1

(57) ABSTRACT

Provided are tubular structures of a biocompatible, triggerably-dissolvable material such as cellulose or a copolymer having an LCST below physiological temperatures. The structures may be embedded within a cell growth scaffold. The tubular structures are useful in growing 3-dimensional tissue structures because nutrients, cytokines or other cell growth and/or differentiation compounds, as well as drugs, such as antibiotics and steroids, can be administered over time, and the tubular structures can be dissolved non-invasively.

### CROSS-REFERENCE TO RELATED APPLICATIONS

This application claims the benefit under 35 U.S.C. § 119(e) to U.S. Provisional Patent Application No. 60/974,922, filed on Sep. 25, 2007, which is incorporated herein by reference in its entirety.

### STATEMENT REGARDING FEDERAL FUNDING

The U.S. Government has a paid-up license in this invention and the right in limited circumstances to require the patent owner to license others on reasonable terms as provided for by the terms of Grant Number T32 HL076124 awarded by the National Institutes of Health.

Provided herein are implantable tubules and cell growth substrates, as well as culturing systems for assisting in tissue growth and regrowth. Also provided are methods of growing tissue in vivo or in vitro, such as for wound healing or other tissue generation/regeneration applications.

Angiogenesis, the growth of new microvessels from parent microvessels is an essential component of new tissue growth, occurring in many physiologic processes including wound healing (Norrby K, In vivo models of angiogenesis. J Cell Mol. Bio., 2006. 10(3): 588-612). Many angiogenic growth factors have been identified and shown to increase angiogenesis in both in vitro and in vivo applications. In order to apply the most effective and productive treatment from these factors, controlled delivery is essential as exogenous delivery can lead to cytotoxicity and growth factor instability (Layman H, et al., The effect of the controlled release of basic fibroblast growth factor from ionic gelatin-based hydrogels on angiogenesis in a murine critical limb ischemic model. Biomaterials, 2007. 28(16): 2646-54). Some researchers have tried to “preprogram” a scaffold to sustainably release growth factors according to a certain schedule but current controlled release technology does not permit adjusting doses to match different tissues, wound sites, or individuals (Ennett A B, Kaigler D, and Mooney D J, Temporally regulated delivery of VEGF in vitro and in vivo. J Biomed Mat Res A, 2006. 79: 176-84).

### SUMMARY

Provided herein is a hollow fiber delivery system capable of providing nutrients, growth factors, therapeutic agents, etc. to a wound site or tissue culture construct. Precise control over delivery from these porous fibers can be used to induce tissue growth, development of native vasculature and osteogenesis, along with other growth and/or differentiation events. To this end, one design factor is a non-invasive mechanism to remove the device following growth of the tissue. The hollow fibers can be dissolved by any physical, chemical or enzymatic process, or combination thereof.

In one non-limiting embodiment, the hollow fibers are manufactured using a water-insoluble polymer that can be solubilized by chemical or enzymatic degradation processes. Cellulose is one non-limiting example of a material for such a device. It is a natural, biocompatible material that can be degraded enzymatically with cellulase. In another non-limiting embodiment, a tough, biodegradable elastomer based on polyurethane and N-isopropylacrylamide (NIPAM) monomers is useful in such a device. This composition has excellent elasticity and strength at body temperature while exhibiting a sharp phase transition to solubility when adjusted to room temperature. Porous hollow fibers fabricated from such materials can serve as an externally-regulated delivery system that can be triggerably-dissolved through a simple change in the properties of the feed, for instance, by lowering the temperature of the feed below the lower critical solution temperature (LCST) of the composition. Of note: 1) cellulose has been proposed as an appropriate biomaterial for other in vivo applications as it can be removed by applying a physiologically non-destructive enzyme (see, Martson M, et al., Biocompatibility of cellulose sponge with bone. Eur Surg Res, 1998. 30(6): 426-432; Muller F A, et al., Cellulose-based scaffold materials for cartilage tissue engineering. Biomaterials, 2006. 27(21): 3955-63; Yang M B, et al. Hollow fibers for hepatocyte encapsulation and transplantation—studies of survival and function in rats. Cell Transplantation, 1994. 3(5): 373-85); 2) synthetic, NIPAM-based material is biodegradable, and possesses attractive mechanical and physical properties; and 3) it has been demonstrated that growth factors can be controllably released using other types of porous, hollow fibers (Kawakami O, et al., Acceleration of aneurysm healing by hollow fiber enabling the controlled release of basic fibroblast growth factor. Neurosurgery, 2006. 58(2): 355-64; Lazzeri L, et al., Biodegradable hollow microfibres to produce bioactive scaffolds. Polymer International, 2005. 54: 101-107; Tilakaratne H K, et al., Characterizing short-term release and neovascularization potential of multi-protein growth supplement delivered via alginate hollow fiber devices. Biomaterials, 2007. 28(1): 89-98).

Thus, according to one embodiment of the present invention, a tubular structure is provided comprising hollow fibers of a biocompatible, triggerably-dissolvable material. The structure is typically has an inside diameter (diameter of the lumen of the tubular structure) of less than 0.1 inches. In one embodiment, the biocompatible, triggerably-dissolvable material comprises a water-insoluble polysaccharide, such as cellulose or cellulose acetate. In additional embodiments, the material has a void fraction of more than about 0.10 or 0.25.

The biocompatible, triggerably-dissolvable material may comprise a copolymer. According to certain embodiments, the copolymer has a lower critical solution temperature of less than 30° C. Examples of such copolymers include copolymers comprising an N-alkyl acrylamide residue in which the alkyl is one of methyl, ethyl, propyl, isopropyl and cyclopropyl; one or both of acrylic acid and methacrylic acid; and an acrylic residue having an amine-reactive group, the copolymer comprising a polyester linkage in its backbone. In one embodiment, the N-alkyl acrylamide is N-isopropylacrylamide. In another, the amine-reactive group is one of a succinimide group, an oxysuccinimide group and an isocyanate group. In yet another, the copolymer comprises an acrylic acid residue. In yet another embodiment, the polymer has the composition (ratios of monomers/macromers in copolymers, such as 85/6/5/4 and 90/0/0, below, are expressed as feed ratios unless otherwise noted):

P(NIPAAm-co-AAc-co-NHS-co-HEMAPLA2.1) 85/6/5/4;

P(NIPAAm-co-AAc-co-NHS-co-HEMAPLA3.9) 85/6/5/4;

P(NIPAAm-co-AAc-co-NHS-co-HEMAPLA7.0) 85/6/5/4;

P(NIPAAm-co-AAc-co-NHS-co-HEMAPLA2.1) 80/6/5/9;

P(NIPAAm-co-AAc-co-NHS-co-HEMAPLA3.9) 80/6/5/9;

P(NIPAAm-co-AAc-co-NHS-co-HEMAPLA2.1) 75/6/5/14;

P(NIPAAm-co-AAc-co-NHS-co-HEMAPLA3.9) 75/6/5/14;

P(NIPAAm-co-AAc-co-NHS-co-HEMAPLA2.1) 85/6/4/4;

P(NIPAAm-co-NHS-co-AAc) 90/0/10;

P(NIPAAm-co-NHS-co-AAc) 90/10/0;

P(NIPAAm-co-NHS-co-AAc) 93.5/0/6.5; or

P(NIPAAm-co-NHS-co-AAc) 93.5/6.5/0.

According to certain embodiments, the copolymer has a lower critical solution temperature above 37° C. after its ester bonds are hydrolyzed so as to render the degradation products of the copolymer soluble at physiological temperatures. The backbone of the polymer may comprise from 1% to 10% ester bonds and may comprise a polyester macromer, such as a polyester macromer comprising hydroxyethyl methacrylate and lactide residues. In one embodiment, the ratio of hydroxyethyl methacrylate to lactide residues in the polyester macromer ranges from 1:1 to 1:10 or from 1:2 to 1:8. In another embodiment, the polyester macromer comprises hydroxyethyl methacrylate and trimethyl carbonate residues, which in one embodiment the ratio of hydroxyethyl methacrylate to trimethyl carbonate residues in the polyester macromer ranges from 1:1 to 1:10 or from 1:2 to 1:5. In yet another embodiment, the copolymer comprises one or more of a caprolactone, a glycolide and a trimethylene carbonate residue.

The copolymer also may comprise an amine-containing compound attached to the copolymer. In one embodiment, the amine-containing compound is one or both of collagen and gelatin. For example, the copolymer may comprise between 1% wt and 10% wt collagen. According to certain embodiments, the material has an elastic modulus of from 0.5 MPa to 1 MPa.

In further embodiments of the tubular structure, a portion of the hollow fibers are embedded in a cell growth scaffold, such as a hydrogel. The scaffold may comprise extracellular matrix-derived material, such as a Matrigel™ or a collagen. The scaffold also may comprise a synthetic copolymer, such as any of the copolymers described herein having an LCST of lower than 37° C.

Also provided herein are methods of delivering a composition to a patient. The methods comprise implanting within the patient a cell growth scaffold comprising a plurality of hollow fibers of a biocompatible, triggerably-dissolvable material, for example and without limitation, the materials described herein, wherein the hollow fibers comprise a first portion embedded in the cell growth scaffold and a second portion extending from the cell growth scaffold; and injecting the composition into the hollow fibers. In one embodiment, the biocompatible, triggerably-dissolvable material comprises a cellulose. In another, the biocompatible, triggerably-dissolvable material comprises a copolymer having a lower critical solution temperature of less than 30° C. The copolymer may be any copolymer described herein for use in preparing hollow fibers. The method further comprises solubilizing (rendering the material soluble in water, e.g., by dropping the temperature of the copolymer below its LCST) or eroding the biocompatible, triggerably-dissolvable material after injecting the composition into the hollow fibers. The composition typically is a liquid that comprises one or more of nutrients, growth factors, cytokines, therapeutic agents, such as antibiotics, etc., such as one or more of VEGF, PDGF, S1P, and bFGF. The hollow fibers may comprise a third portion that extends from the scaffold that is opposite the second portion. The hollow fibers typically have an inside diameter of less than 0.1 inches.

Also provided is a cell culture apparatus comprising a culture vessel containing a tubular structure, for example and without limitation, as described above, the tubular structure either extending outside the culture vessel or being connected to a fluid connector that extends a fluid path from the tubular structure to outside the culture vessel. The cell culture apparatus may comprise a fluid connector fluidly connected to the tubular structure that extends a fluid path from the tubular structure to outside the culture vessel, the fluid connector comprising a valve. The cell culture apparatus may further comprise a cell growth scaffold in which the tubular structure is partially embedded (a portion of the tubular structure is embedded) within the culture vessel. In a related embodiment, a method of culturing cells or tissue, comprising culturing cells in the cell culture apparatus is provided.

According to yet another embodiment, a method of growing tissue is provided, the method comprises in a cell culture apparatus or in vivo, contacting cells with a cell growth scaffold comprising a plurality of hollow fibers of a biocompatible, triggerably-dissolvable material, for example and without limitation, as described herein, comprising a first portion embedded in the cell growth scaffold and a second portion extending from the cell growth scaffold, and administering one or more of a cell growth nutrient, a growth factor and a therapeutic agent to the scaffold through the tubular structure. The method also may further comprise dissolving all or part of the tubular structure after administering the one or more of a cell growth nutrient, a growth factor and a therapeutic agent to the scaffold through the tubular structure.

### BRIEF DESCRIPTION OF THE DRAWINGS

FIG. 1 shows schematically an in vitro setup (one well of a six well plate). In this non-limited embodiment, the fiber is embedded in Matrigel™ and set in agarose.

FIGS. 2A-B (“Prior Art”) show cellulose acetate hollow fibers for three-dimensional tissue engineering (taken from Ko I K and Iwata H. Ann NY Acad. Sci., 2001. 944: 443-55). FIG. 2A shows the hollow fibers before cellulase treatment. FIG. 2B shows the hollow fibers after cellulase mediated degradation, leaving only endothelial cells.

FIG. 3 (“Prior Art”) shows PLLA hollow fibers coated with PLGA microspheres (taken from Lazzeri L, et al., Polym Int., 2005. 54: 101-107). The graph shows the release profile of dextromethasone (“DXM”) in vitro from microspheres (curve on graph labeled with “(a)”) and from hollow fibers coated with microspheres (curve on graph labeled with “(b)”). The inset shows a scanning electron micrograph of a particle coated fiber.

FIG. 4 (“Prior Art”) is a graph showing endothelial cell growth supplement (“ECGS”) release from polypropylene hollow fibers in an in vitro setting (taken from Tilakaratne H K, et al., Biomaterials, 2007. 28(1): 89-98).

FIGS. 5A-C (“Prior Art”) show photomicrographs of Masson trichrome-stained vessel implants three weeks following induced aneurysm (original magnification at 20×, taken from Kawakami O, et al., Neurosurgery, 2006. 58(2): p. 355-64). FIG. 5A shows an untreated vessel, FIG. 5B shows an empty polyethylene hollow fiber, and FIG. 5C shows a polyethylene hollow fiber loaded with a bFGF collagen gel. “AL” indicates arterial lumen, “FT” indicates fibrous tissue, and scale bars are 1 mm.

FIG. 6 shows a schematic of an experimental setup for hollow fiber fabrication, where nominal inner diameter (“I.D.”) is provided in inches.

FIGS. 7A-D are scanning electron micrographs of hollow fibers. Micrographs are shown for cellulose acetate fibers, where FIG. 7A shows the fiber walls and FIG. 7C shows the pore structure. Micrographs are shown for NIPAM-based polymer fibers, where FIG. 7B shows the fiber walls and FIG. 7D shows the pore structure.

FIGS. 8A-C are scanning electron micrographs of cellulose hollow fibers with various water flow rates. FIG. 8A shows the pore structure for a flow rate of 10 mL/min, where void fraction is 0.115. FIG. 8B shows the pore structure for a flow rate of 15 mL/min, where void fraction is 0.125. FIG. 8C shows the pore structure for a flow rate of 20 mL/min, where void fraction is 0.241.

FIGS. 9A-B are scanning electron micrographs of cellulose fibers with various cellulose flow rate. FIG. 9A shows the pore structure for a flow rate of 1.5 mL/min, where void fraction is 0.125. FIG. 9B shows the pore structure for a flow rate of 2.0 mL/min, where void fraction is 0.286.

FIG. 10A-C are graphs showing the release from hollow fibers into saline supernatant. FIG. 10A shows the release of fluorescein (MW=330), FIG. 10B shows the release of cytochrome c (MW=12,000), and FIG. 10C shows the release of bovine serum albumin (MW=66,000).

FIG. 11 is a graph showing the release of bovine serum albumin (“BSA”) from high interconnectivity fibers. Release profiles are shown for different concentrations of BSA (50, 150, and 200 mg/mL) in the lumen of the fibers.

FIG. 12 is a graph showing the release of fluorescein from high interconnectivity fibers embedded in Matrigel™.

FIGS. 13A-B are scanning electron micrographs of cellulose hollow fibers. FIG. 13A shows a cross section of the fiber wall at 850× magnification. FIG. 13B shows the microporous structure at 10,000× magnification.

FIGS. 14A-F are phase/contrast photomicrographs of tubular formation in human umbilical vein endothelial cells (HUVECs) plated on Matrigel™ with or without sphingosine-1-phosphate (“S1P”). Photomicrographs are shown for HUVECs treated with no fiber and M199 (FIG. 14A); M199 injected into fiber (FIG. 14B); no fiber, 5 μM S1P in M199 (FIG. 14C); 600 mM S1P injected into fiber (FIG. 14D); 1200 mM S1P injected into fiber (FIG. 14E); and 1800 mM S1P injected into fiber (FIG. 14F).

FIG. 15 is a fluorescence photomicrograph of rhodamine phalloidin-stained HUVECs plated on Matrigel™, indicating the presence of F-actin.

FIG. 16 is a graph showing the percent area covered by HUVECs plated on Matrigel™. Data is shown for HUVECs treated with no fiber and M199 (labeled “A”); M199 injected into fiber (labeled “B”); no fiber, 5 μM S1P in M199 (labeled “C”); 600 mM S1P injected into fiber (labeled “D”); 1200 mM S1P injected into fiber (labeled “E”); and 1800 mM S1P injected into fiber (labeled “F”). Percentage area covered by HUVECs plated on Matrigel™ was calculated by using threshold analysis on F-actin (rhodamine phalloidin) stained images.

FIGS. 17A-B are photomicrographs of HUVECs at day 3 (FIG. 17A) and at confluency (FIG. 17B).

FIG. 18 is a table showing a non-limiting example of an experimental design for testing delivery of S1P to HUVECs.

FIG. 19 is a schematic of an artificial wound capillary (wound cap) bed, which is described herein.

FIGS. 20A-D are brightfield photomicrographs of HUVECs in an in vitro scrape wound assay.

FIGS. 20A and 20C shows the confluent layers of HUVECs after the scrape. FIG. 20B shows the cell layer 42 hours after the scrape with media injected into the hollow fiber immersed in media. FIG. 20D shows the cell layer 42 hours after the scrape with 1800 μM S1P injected into the hollow fiber immersed in media.

FIGS. 21A-H are photomicrographs of Matrigel™ plug explants 7 days post-implantation in mice (200× magnification). FIGS. 21A-D show immunostained photomicrographs of H&E-stained sections of Matrigel™ plug explants and FIGS. 21E-H shows immunofluorescence photomicrographs of CD31-stained sections of Matrigel plug explants. Hollow fibers were injected with VEGF (FIGS. 21A, 21E); with S1P (FIGS. 21B, 21F); with VEGF followed by S1P (FIGS. 21C, 21G); and with S1P followed by VEGF (FIGS. 21D, 21H).

FIG. 22 is a graph showing percent area measurement of endothelial cells in Matrigel™ plug explants.

FIG. 23 is a schematic diagram showing the stages of bone regeneration.

FIG. 24 is a schematic of a cross-sectional view of the wall of the urinary bladder (not drawn to scale). The following structures are shown: epithelial cell layer (A), basement membrane (B), tunica propria (C), muscularis mucosa (D), tunica submucosa (E), tunica muscularis extema (F), tunica serosa (G), tunica mucosa (H), and the lumen of the bladder (L).

FIG. 25 is a schematic of a cross-sectional view of a non-limiting example of a cell culture apparatus comprising hollow fibers.

### DETAILED DESCRIPTION

As used herein, the terms “comprising,” “comprise” or “comprised,” and variations thereof, in reference to elements of an item, composition, apparatus, method, process, system, etc. are meant to be open-ended.

Also described herein are methods, compositions and products for use in tissue repair, regeneration, generation etc. For example and without limitation, the data described above describes compositions of matter in the form of triggerably-dissolvable porous hollow fibers and its reduction to practice in the form of controlled release and inducing endothelial cells to form vessel-like structures in extracellular matrix. To our knowledge, we are the first investigators to use a triggerably-dissolvable hollow fiber for the delivery of growth factors and other therapeutic agents in the context of tissue engineering. According to one embodiment, a device is provided comprising an array of hollow fibers that can be implanted into a wound site for the controlled delivery of site-specific growth factors in order to facilitate healing by controlling the cellular response at that site.

As used herein, a “polymer” is a compound formed by the covalent joining of smaller molecules, which are referred to herein as residues, or polymer subunits, when incorporated into a polymer. A “copolymer” is a polymer comprising two or more different residues. Prior to incorporation into a polymer, the residues typically are described as monomers. Non-limiting examples of monomers, in the context of the acrylic/polyester copolymer described herein, include: acrylic or acrylamide monomers, such as acrylic acid, acrylic N-hydroxysuccinimide ester and hydroxyethyl methacrylate, lactide, and trimethylene carbonate. A monomer may be a macromer prepared from even smaller monomers, such as the hydroxyethyl methacrylate-polylactide (HEMAPLA) macromer or the hydroxyethyl methacrylate-poly(trimethylene carbonate) (HEMAPTMC) macromer described herein. As used herein, an acrylic monomer has the general structure (CH₂═CH—C(O)—R), and, when polymerized, forms the general polymer structure having an alkylene backbone ( . . . C—C—C—C—C . . . ) and the overall structure: . . . C—(—C(C(O)R)—C—)ₙ—C(C(O)R)—C . . . in which each instance of R can be the same, or in the case of a copolymer, independently different.

The term “polymer” refers to both synthetic polymeric components and biological polymeric components. “Biological polymer(s)” are polymers that can be obtained or derived from biological sources, such as, without limitation, mammalian or vertebrate tissue, as in the case of certain extracellular matrix-derived (ECM-derived) compositions, such as collagens. In another non-limiting example, the biological polymer is derived from a plant source, such as cellulose. Biological polymers can be modified by additional processing steps. Polymer(s), in general include, for example and without limitation, mono-polymer(s), copolymer(s), polymeric blend(s), block polymer(s), block copolymer(s), cross-linked polymer(s), non-cross-linked polymer(s), polysaccharide(s), linear-, branched-, comb-, star-, and/or dendrite-shaped polymer(s).

As used herein, “collagen” refers to one or more different types of collagen that is derived from synthetic and/or natural sources. For example and without limitation, collagen can be derived from the extracellular matrix or connective tissue. Collagen exists in many different forms. For example and without limitation, these forms include fibrillar collagens, such as Type I, II, III, and IV; fibril-associated collagens, such as Type VI and IX; and sheet-forming collagen, such as Type IV.

As used herein, “cellulose” refers to cellulose and derivatives thereof. Cellulose is a polysaccharide with the general repeating unit comprising linked D-glucose units. Derivatives of cellulose include those modifications to the hydroxyl groups of cellulose, such as, without limitation, cellulose esters and cellulose ethers. Examples of cellulose esters include, without limitation, cellulose acetate, cellulose triacetate, (acrylamidomethyl)cellulose acetate propionate, cellulose acetate butyrate, and cellulose acetate propionate. Example of cellulose ethers include, without limitation, cyanoethylated cellulose, ethyl cellulose, 2-hydroxyethyl cellulose, hydroxypropyl cellulose, hydroxyethylcellulose ethoxylate, (hydroxypropyl)methyl cellulose, methylcellulose, carboxymethyl cellulose, and methyl 2-hydroxyethyl cellulose.

In one embodiment, the hollow fibers are prepared from an enzymatically degradable material, such as a polysaccharide that is water-insoluble until it is eroded or digested enzymatically or chemically. The polysaccharide and its degradation products are biocompatible, for example and without limitation, they are not cytotoxic. The polysaccharide also degrades and dissolves at physiological conditions in a time-dependent manner, which is important for removal of the polysaccharide after the applied surgical or medical procedure. Polysaccharides are available in many forms, such as in solution, as microcrystalline powders, microgranular powders, fibers, colloidal powder, and sheets. According to one embodiment, the polysaccharide comprises cellulose, which either is available in solution or is mixed with a solvent to create a solution. Hollow fibers can be obtained by extruding the solution containing the polysaccharide and then precipitating the polysaccharide. The hollow fibers (or filaments) can be triggerably-dissolved by injecting them with a solution containing enzymes that degrade the polysaccharide. Enzymes to degrade cellulose include, for example and without limitation, glycoside hydrolases, such as cellulose and glucosidase.

In another embodiment, the hollow fibers are prepared from a thermoresponsive and biodegradable elastomeric material, namely a copolymer. The copolymer remains fluid at and below room temperature, solidifies at physiological temperature, and binds to biological molecules. The copolymer also degrades and dissolves at physiological conditions in a time-dependent manner, which is important for removal of the copolymer after the applied surgical or medical procedure. The copolymer and its degradation products are biocompatible, for example and without limitation, they are not cytotoxic. The synthetic copolymer has a lower critical solution temperature (LCST) below physiologic temperatures so that it is a solid at physiologic temperatures and dissolves at lower temperatures, such as less than 35° C. and preferably less than 30° C., 27° C. or 25° C. Non-limiting examples of suitable copolymers are provided in United States Patent Publication No. 2008-0096975 A1. According to one embodiment, the copolymer comprises an N-isopropylacrylamide residue (an N-isopropylacrylamide monomer incorporated into a polymer), one or both of an acrylic acid residue and a methacrylic acid residue and an acrylic residue having an amine-reactive group. The copolymer comprises a polyester linkage in its backbone. According to one non-limiting embodiment, the copolymer is prepared from at least five components: N-isopropylacrylamide or an N-alkyl acrylamide in which the alkyl is methyl, ethyl, propyl, isopropyl or cyclopropyl, acrylic acid and/or methacrylic acid, an acrylic monomer having an amine-reactive group (such as acrylic N-hydroxysuccinimide ester), collagen and a polyester macromer. For example and without limitation, the polyester macromer is a polylactide macromer, comprising hydroxyethyl methacrylate residues and varying numbers of lactide units/residues. In another non-limiting example, the polyester macromer is a poly(trimethylene carbonate macromer), comprising hydroxyethyl methacrylate residues and varying numbers of trimethylene carbonate units/residues. Each component contributes to the desired physical properties of the hydrogel to enable a material that can be formed into hollow filaments that may be implanted into a patient for delivering drugs or chemicals, encapsulating and transplanting cells, and injecting into empty cavities for wounds or tissue repair. The hollow filaments can be triggerably-dissolved by either flushing them with a solution below the LCST of the filaments, or otherwise cooling the filaments. The amine-reactive component of the copolymer (for instance, acrylic N-hydroxysuccinimide ester) can bind to amine-containing compounds including biomolecules such as collagen and/or other bioactive or biocompatible materials or factors in order to facilitate ingrowth of tissue about the hollow filaments.

The composition of each component in the copolymer determines the lower critical solution temperature (LCST), the strength and elasticity of the copolymer. As a solid, the copolymer is highly flexible and relatively strong at physiological temperature. At a temperature less than the LCST, the hydrogel flows easily and loses its shape.

A polyester component within the macromer introduces the degradability and hydrophobicity of the copolymer. For complete degradation of the copolymer during, and especially after controlled melting, the copolymer includes hydrolytically-cleavable bonds that results in soluble, non-toxic by-products, even above the LCST of the non-degraded copolymer. Once the copolymer is degraded, the LCSTs of the degradation products are above physiological temperature, which results in dissolution of the degraded hydrogel and clearance of the degraded components.

In further detail, according to one non-limiting embodiment, the copolymer comprises an N-alkyl acrylamide residue in which the alkyl is methyl, ethyl, propyl, isopropyl or cyclopropyl, such as N-isopropylacrylamide residue, one of an acrylic acid residue and a methacrylic acid residue, and an acrylic residue having an amine-reactive group. The copolymer comprises a biodegradable polyester linkage in its backbone. In one non-limiting embodiment, the amine-reactive group is a succinimide group, an oxysuccinimide group or an isocyanate group. In one embodiment, the copolymer has a lower critical solution temperature below 37° C., in another between 30° C. and 34° C. and in another, less than 27° C. According to one embodiment, the copolymer has a lower critical solution temperature above 37° C. after its ester bonds are hydrolyzed.

To facilitate hydrolysis of the copolymer, according to one embodiment, the backbone of the copolymer comprises biodegradable ester linkages, for example and without limitation, from 1% to 10% of the linkages of the copolymer backbone. The number of polyester groups can be altered in order to control the rate of degradation of the copolymer. In the context of hollow filaments used to feed tissue, the tubes may be dissolved, leaving solid “lumps” of the copolymer, which will degrade over time. Because the copolymer is suitable for use as a cell growth scaffolding, as the copolymer erodes, it will support in-growth of tissue as a final step in the tissue growth/repair process.

The copolymer may comprise a polyester macromer, for example and without limitation, a polyester macromer comprising hydroxyethyl methacrylate and lactide residues. In one embodiment, the ratio of hydroxyethyl methacrylate and lactide residues in the polyester macromer is from 1:2 to 1:8, in another, from 1:1 to 1:10, such as 1:1, 1:2, 1:3, 1:4, 1:5, 1:6, 1:7, 1:8, 1:9, and 1:10. In another non-limiting example, the polyester macromer comprises hydroxyethyl methacrylate and trimethylene carbonate residues. In one embodiment, the ratio of hydroxyethyl methacrylate and trimethylene carbonate residues in the polyester macromer ranges from 1:1 to 1:10, 1:2 to 1:5 or any increment within those ranges, including 1:1, 1:2, 1:3, 1:4, 1:4.2, 1:5, 1:6, 1:7, 1:8, 1:9, and 1:10. Amine-containing biomolecules or other compounds, such as proteins, carbohydrates, glycoproteins, etc. can be conjugated to the copolymer through the amine-reactive group. In certain embodiments, collagen, heparin or gelatin are suitable compounds, for instance and without limitation, between 1% wt and 10% wt collagen. In one embodiment, the copolymer comprises caprolactone, glycolide or trimethylene carbonate residues.

In one embodiment of the copolymer useful in humans or animals, the copolymer has a lower critical solution temperature below 37° C. For veterinary applications, the LCST can be slightly higher as the core body temperature of certain animals (e.g., cats, dogs, horses, cows, sheep and goats) is in the range of 38° C.-39° C. In another embodiment, the copolymer has a lower critical solution temperature above 37° C. after its backbone ester linkages are hydrolyzed (substantially hydrolyzed, as with treatment of the polymer with NaOH, as described herein).

The thermosensitive copolymer can be manufactured by any suitable method, for example and without limitation, the method comprises co-polymerizing N-isopropylacrylamide, acrylic acid and/or methacrylic acid, an acrylic monomer having an amine-reactive group and a polyester linkage-containing monomer to make a copolymer comprising an acrylic and polyester backbone. The monomers can be co-polymerized by any useful polymerization method, for example and without limitation by free-radical polymerization. In one instance, the polyester linkage-containing monomer is a polyester macromer, for example and without limitation, prepared from hydroxyethyl methacrylate and lactide. In one embodiment, the ratio of hydroxyethyl methacrylate and lactide residues in the polyester macromer is from 1:2 to 1:8 or from 1:1 to 1:10. In another non-limiting example, the polyester macromer is prepared from hydroxyethyl methacrylate and trimethylene carbonate. In another instance, the polyester linkage-containing monomer is one of a caprolactone, a glycolide and a trimethylene carbonate monomer.

According to certain embodiments, the copolymers comprise four types or subunits/residues: 1) N-alkyl acrylamide in which the alkyl is methyl, ethyl, propyl, isopropyl or cyclopropyl, for example N-isopropylacrylamide, as a thermosensitive component after polymerization; 2) acrylic acid N-hydroxysuccinimide ester for conjugation of biomolecules; 3) acrylic acid for improvement of hydrophilicity and 4) polyester macromer for introduction of degradability and hydrophobicity. The hydrophobic units of the polyester macromer, for example and without limitation, the lactide units or trimethylene carbonate units, decrease the LCST of the copolymer to well below 37° C. before degradation. After degradation, it forms hydrophilic poly(hydroxyethyl methacrylate) structure in the backbone, which increases hydrophilicity of the polymer, LCST is then increased to above 37° C.

The materials, copolymers, compositions and components thereof are preferably biocompatible. By “biocompatible,” it is meant that a polymer composition and its normal in vivo degradation products are cytocompatible and are substantially non-toxic and non-carcinogenic in a patient within useful, practical and/or acceptable tolerances. By “cytocompatible,” it is meant that the copolymers or compositions are substantially non-toxic to cells and typically and most desirably can sustain a population of cells and/or the polymer compositions, devices, copolymers, and degradation products thereof are not cytotoxic and/or carcinogenic within useful, practical and/or acceptable tolerances. For example, a copolymer composition when placed in a human epithelial cell culture does not adversely affect the viability, growth, adhesion, and number of cells. In one non-limiting example, the co-polymers, compositions, and/or devices are “biocompatible” to the extent they are acceptable for use in a human veterinary patient according to applicable regulatory standards in a given legal jurisdiction. In another example the biocompatible polymer, when implanted in a patient, does not cause a substantial adverse reaction or substantial harm to cells and tissues in the body, for instance, the polymer composition or device does not cause necrosis or an infection resulting in harm to tissues organs or the organism from the implanted compositions.

Polyester polymer backbones are polymer backbones containing two or more ester groups. A polyester backbone has an average of more than one ester units (—C(O)O—), as opposed to an ester linkage that has one ester unit. An example is a polylactide macromer as described herein. Another example is a poly(trimethylene carbonate) macromer. Other examples of residues that comprise ester linkages include, without limitation, caprolactones, glycolides and a trimethylene carbonate residues.

Polyester macromers are compounds containing on the average one or more, and preferably two or more ester linkages. In the context of macromer and polymer preparations, unless otherwise indicated, the number of residues indicated as being present in a given polymer or macromer is an average number and is not to be construed as an absolute number. Thus, as a non-limiting example, in the context of the HEMAPLA macromers, the numbers 2.1, 3.9 and 7.0 refer to an estimated average number of —C(O)—C(CH₃)—O— residues present in the macromers in the macromer composition, and, when incorporated into a copolymer, the average number of —C(O)—C(CH₃)—O— residues present in the incorporated polyester macromer residues, for example as shown in the Examples below. The average number of residues may be determined by any method, for example and without limitation, by 1H-NMR, as in the examples, below.

Lower critical solution temperature (LCST) refers to the temperature below which the constituents of the hydrogel are soluble in water and above which the constituents are insoluble. When the LCST is reached, the polymer constituents in an aqueous solution will aggregate to form hydrogel. The LCST can be determined by measuring the change in transmittance with a UV-Vis spectrometer as a function of temperature (Ron E S and Bromberg L E, Temperature-responsive gels and thermogelling polymer matrices for protein and peptide delivery. Adv Drug Deliv Rev. 1998 May 4; 31(3): 197-221 and Healy K E et al., Designing biomaterials to direct biological responses. Ann N.Y. Acad. Sci. 1999 Jun. 18; 875(1):24-35). LCST also can be determined by any other useful method—for example and without limitation by Differential Scanning Calorimetry (DSC). DSC is used to measure LCTS in the examples below.

One unique aspect of the copolymers described herein is that the LCST of these copolymers is typically between 18° C. and about 37° C. One limitation in the production and distribution of structures manufactured from the copolymers is that to retain the structure, the temperature of the copolymer must be maintained above the LCST the entire time between manufacture and implantation. This may be accomplished in the manufacture/distribution chain by any suitable means, including the use of heated manufacturing and storage facilities, heat packs and insulated packaging. Alternately, the copolymer filaments may be manufactured at the site of use, provided there is a suitable facility and equipment for doing so. The logistics and preferred methods for manufacturing, storing and transporting the copolymer devices described herein are within the abilities of those of skill in the art.

In one embodiment, the copolymer comprises an N-isopropylacrylamide residue, one or both of an acrylic acid residue and a methacrylic acid residue (CH₂═C(CH₃)C(O)OH, an acrylic residue having an amine-reactive group, the copolymer having polyester linkages in its backbone. The copolymer may be reacted with amine-containing compositions, such as compositions or molecules comprising amine groups, for example and without limitation, collagen, fibrin, gelatin and heparin. The polyester linkages may be incorporated in the copolymer backbone by introduction of, for example and without limitation, one or more of a polyester macromer, a polycaprolactone, a polyglycolide and a poly(trimethylene carbonate) into the copolymer. As described above, in certain non-limiting examples, the polyester linkages are introduced into the copolymer as a polyester macromer, such as a macromer comprising hydroxyethyl methacrylate and lactide residues. Monomers (including as a group macromers) containing ester linkages can be introduced into the copolymer by radical polymerization, or in any useful manner using any suitable initiator, such as benzoyl peroxide.

Amine-reactive groups are groups that react with amine residues, such as Lys residues of proteins, to form a covalent linkage. Non-limiting examples of amine-reactive groups are succinimide, oxysuccinimide or isocyanate groups. A non-limiting examples of a useful acrylic monomer includes an N-acryloxysuccinimide (NHS) ester, having the structure:

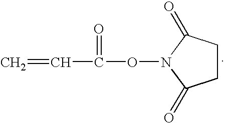

Non-limiting examples of suitable co-polymers are described in United States Patent Publication No. 2008-0096975 A1. In one non-limiting embodiment, a suitable co-polymer is prepared first by synthesizing a suitable polyester macromer and then by incorporating the macromer into a larger co-polymer. For example and without limitation, polylactide macromer HEMAPLA can be synthesized by ring-open polymerization of lactide with 2-hydroxyethyl methacrylate with stannous octoate as catalyst. Stoichiometric amounts of lactide and 2-hydroxyethyl methacrylate (HEMA) can be mixed in a three-neck flask. Stannous octoate (e.g., 1 mol % with respect to HEMA) in, e.g., 1 mL toluene can then be added. Reaction can be conducted at 110° C. for 1.5 hours under a nitrogen atmosphere. The mixture can then be dissolved in tetrahydrofuran (THF) and precipitated in cool water. The precipitate can be collected by centrifugation, dissolved in ethyl acetate and dried with MgSO₄ overnight. Ethyl acetate can be evaporated under reduced pressure. The yield, a viscous oil can then be dried in a vacuum oven overnight. Polylactide macromers with various lactide units can be synthesized by altering feed ratio of lactide and HEMA. In one example, polylactide macromers with lactide units 2.1, 3.9 and 7.0 (the average number of lactide units per “macromer”) were obtained with lactide:HMEA feed ratios 1, 2 and 4, respectively. The content of the macromer and copolymer may be confirmed by Fourier Transform Infrared spectrum (FT-IR).

P(NIPAAm-co-AAc-co-NHS-co-HEMAPLA) copolymer, which is composed of N-isopropylacrylamide (NIPAAm), acrylic acid (AAc), acrylic N-hydroxysuccinimide ester (NHS), and HEMAPLA macromer can be synthesized, for example and without limitation, by radical polymerization with benzoyl peroxide (BPO) as initiator. Stoichiometric amount of monomers can be dissolved in 1,4-dioxane and mixed in a 250 mL one-neck flask under an argon atmosphere for 10 minutes. Degassed BPO (7.2×10⁻³ mol/mol monomer) in 1,4-dioxane solution is then added into the flask. The reaction is carried out at 70° C. for 24 hours. The mixture is cooled to room temperature, precipitated in hexane and filtered. The polymer is dried overnight in a vacuum oven and then purified by repeating dissolving in THF and precipitating with diethyl ether. Table 1 provides the feed ratios and composition (as determined by 1H-NMR) for exemplary copolymers. Structure can be confirmed by 1H-NMR.

TABLE 1
Composition of Poly(NIPAAm-co-NHS-co-AAc-co-HEMAPLA)

| Polymer | Feed ratio | Composition* |
|---|---|---|
| P(NIPAAm-co-AAc-co-NHS-co- | 85/6/5/4 | 85/6.7/3.9/4.4 |
| HEMAPLA2.1**) | | |
| P(NIPAAm-co-AAc-co-NHS-co- | 85/6/5/4 | 85/6.9/4.0/4.1 |
| HEMAPLA3.9**) | | |
| P(NIPAAm-co-AAc-co-NHS-co- | 85/6/5/4 | 85/6.9/3.8/4.3 |
| HEMAPLA7.0**) | | |
| P(NIPAAm-co-AAc-co-NHS-co- | 80/6/5/9 | 80/7.5/4.2/8.3 |
| HEMAPLA2.1**) | | |
| P(NIPAAm-co-AAc-co-NHS-co- | 80/6/5/9 | 80/7.0/4.4/8.6 |
| HEMAPLA3.9**) | | |
| P(NIPAAm-co-AAc-co-NHS-co- | 75/6/5/14 | 75/7.3/4.7/13.0 |
| HEMAPLA2.1**) | | |
| P(NIPAAm-co-AAc-co-NHS-co- | 75/6/5/14 | 75/6.3/4.9/13.8 |
| HEMAPLA3.9**) | | |
| P(NIPAAm-co-AAc-co-NHS-co- | 80/11/5/4 | 80/11.4/4.2/4.4 |
| HEMAPLA2.1**) | | |
| P(NIPAAm-co-AAc-co-NHS-co- | 80/11/5/4 | 80/10.6/6.2/3.2 |
| HEMAPLA3.9**) | | |

Determined by H1-NMR.
**Lactide units

Amine-reactive groups, such as NHS residues can be reacted with collagen or other amine group-containing biomolecules by, for example and without limitation, incubating the mixture overnight at 4° C. Table 2 shows examples of hydrogels that may be prepared.

TABLE 2
Hydrogel formation with collagen.

Collagen content (%)

| Polymer | 0 | 5 | 10 | 20 |
|---|---|---|---|---|
| P(NIPAAm-co-AAc-co-NHS-co- | + | + | + | − |
| HEMAPLA2.1)85/6/5/4 | | | | |
| P(NIPAAm-co-AAc-co-NHS-co- | + | + | + | − |
| HEMAPLA3.9)85/6/5/4 | | | | |
| P(NIPAAm-co-AAc-co-NHS-co- | + | + | + | − |
| HEMAPLA7.0)85/6/5/4 | | | | |
| P(NIPAAm-co-AAc-co-NHS-co- | + | + | + | − |
| HEMAPLA2.1)80/6/5/9 | | | | |
| P(NIPAAm-co-AAc-co-NHS-co- | + | + | + | − |
| HEMAPLA3.9)80/6/5/9 | | | | |
| P(NIPAAm-co-AAc-co-NHS-co- | + | + | + | − |
| HEMAPLA2.1)75/6/5/14 | | | | |
| P(NIPAAm-co-AAc-co-NHS-co- | + | + | + | − |
| HEMAPLA3.9)75/6/5/14 | | | | |
| P(NIPAAm-co-AAc-co-NHS-co- | + | + | + | − |
| HEMAPLA2.1)80/11/5/4 | | | | |
| P(NIPAAm-co-AAc-co-NHS-co- | + | + | + | − |
| HEMAPLA3.9)80/11/5/4 | | | | |

“+” Gelation (form hydrogel), “−” Precipitation (not form hydrogel).

The temperature at the maxima of the endotherm peak can be taken as the LCST. As shown in US Patent Publication No. 2008-0096975 A1, LCSTs for a number of these copolymers were determined for polymers after synthesis and after hydrolysis with NaOH, as shown in Tables 3 and 4. After synthesis, copolymer solutions were formed by dissolving copolymers in PBS (pH=7.4) at 20 wt %. LCSTs of completely hydrolyzed copolymers were measured after hydrolysis in a 1.0 M NaOH solution at 4° C. for 10 days, followed by neutralization with a 10 M HCl solution.

TABLE 3
LCST (in ° C.) for copolymers of various composition ratios

| | After | After NaOH |
|---|---|---|
| Polymer | synthesis | hydrolysis |
| P(NIPAAm-co-AAc-co-NHS-co- | 26.0 | 41.1 |
| HEMAPLA2.1)85/6/5/4 | | |
| P(NIPAAm-co-AAc-co-NHS-co- | 25.8 | 41.4 |
| HEMAPLA3.9)85/6/5/4 | | |
| P(NIPAAm-co-AAc-co-NHS-co- | 24.8 | 42.3 |
| HEMAPLA7.0)85/6/5/4 | | |
| P(NIPAAm-co-AAc-co-NHS-co- | 24.0 | 41.8 |
| HEMAPLA2.1)80/6/5/9 | | |
| P(NIPAAm-co-AAc-co-NHS-co- | 21.0 | 42.0 |
| HEMAPLA3.9)80/6/5/9 | | |
| P(NIPAAm-co-AAc-co-NHS-co- | 19.4 | 41.2 |
| HEMAPLA2.1)75/6/5/14 | | |
| P(NIPAAm-co-AAc-co-NHS-co- | 18.4 | 40.8 |
| HEMAPLA3.9)75/6/5/14 | | |
| P(NIPAAm-co-AAc-co-NHS-co- | 26.1 | 60.6 |
| HEMAPLA2.1)80/11/5/4 | | |
| P(NIPAAm-co-AAc-co-NHS-co- | 26.0 | 61.1 |
| HEMAPLA3.9)80/11/5/4 | | |

TABLE 4
LCST (in ° C.) for copolymers with various collagen content

| | Collagen content (%) | | |
|---|---|---|---|
| Polymer | 0 | 5 | 10 |
| P(NIPAAm-co-AAc-co-NHS-co-HEMAPLA3.9) | 21.0 | 21.2 | 22.4 |
| 80/6/5/9 | | | |

Another suitable copolymer is a poly(NIPAAm-co-NHS) hydrogel. This copolymer is similar to the copolymer described above in terms of LCST, strength and elasticity, but possessing a different degradation mechanism due to its lack of ester linkages in the backbone. The polymer is a copolymer of N-isopropylacrylamide and acrylic N-hydroxysuccinimide ester. The N-isopropylacrylamide serves as the thermosensitive component after polymerization and the acrylic N-hydroxysuccinimide ester is for conjugation of biomolecules. A copolymer of N-isopropylacrylamide and acrylic acid is used as control. This copolymer is synthesized by BOP-initiated radical polymerization substantially as described above. Table 5 provides exemplary monomer feed ratios and composition of the resultant copolymer as determined by 1H-NMR.

TABLE 5
Synthesis of Poly(NIPAAm-co-NHS-co-AAc)

| | Feed ratio | Composition |
|---|---|---|
| | (NIPAAm/ | (NIPAAm/ |
| Polymer | NHS/AAc) | NHS/AAc)* |
| P(NIPAAm-co-NHS-co-AAc) = 90/0/10 | 90:0:10 | 90.2:0:9.8 |
| P(NIPAAm-co-NHS-co-AAc) = 90/10/0 | 90.0:10.0:0 | 88.7:11.3:0 |
| P(NIPAAm-co-NHS-co-AAc) = 93.5/0/6.5 | 93.5:0:6.5 | 92.7:0:7.3 |
| P(NIPAAm-co-NHS-co-AAc) = 93.5/6.5/0 | 93.5:6.5:0 | 92.1:7.9:0 |
| P(NIPAAm-co-NHS-co-AAc) = 80/20/0 | 80.0:20.0:0 | 80.3:19.7:0 |

Determined by 1H-NMR

The hollow fibers can be arranged within a scaffold. As used herein, a “scaffold” or “cell growth scaffold” refers to an arrangement of materials that support cell growth and typically comprises natural and/or synthetic polymers and optionally nutrients, proteins, enzymes, cytokines, therapeutic agents, etc. The scaffold may comprise hollow fibers and can be in any arrangement to facilitate controlled delivery of compositions to the scaffold. In one embodiment, the scaffold comprises a single layer (or array) of parallel hollow fibers. In another embodiment, the scaffold comprises one or more layers of parallel hollow fibers. In yet another embodiment, the scaffold comprises a first layer of parallel hollow fibers and second layer of parallel hollow fibers, wherein the fibers in the first layer are orthogonal to the fibers in the second layer. In further embodiment, the scaffold comprises multiple layers of hollow fibers, wherein the fibers in the uneven numbered layers (first, third, fifth, etc. layers) are orthogonal to the fibers in the even numbered layers (second, fourth, sixth, etc. layers).

To allow for controlled delivery of therapeutic agents, at least one end of the fibers should be accessible for injecting or delivering therapeutic agents. In a certain non-limiting embodiment, the hollow fibers are embedded at one end in a cell growth scaffold. In another, the hollow fibers pass through the cell growth scaffold. The cell growth scaffold can be formed in many different ways. For example and without limitation, the hollow fibers are embedded within the cell growth scaffold to maintain the geometric arrangement of the fibers. For example and without limitation, the scaffold can be formed by casting a cell growth scaffold around the arranged fibers, wherein the cell growth scaffold is then cured or hardened. In another non-limiting example, the cell growth scaffold is injected or implanted at a site within the patient and then the hollow fibers are threaded through the cell growth scaffold. For use within a patient, the cell growth scaffold comprises proteins and/or polymeric constituents that degrade and dissolve at physiological conditions in a time-dependent manner, where the proteins and/or polymers and their degradation products are biocompatible and/or not cytotoxic. The cell growth scaffold can be degraded and/or dissolved by any temperature, chemical and/or biochemical trigger.

In one non-limiting example, the cell growth scaffold comprises a thermoresponsive and biodegradable elastomeric material, namely a copolymer (for example, a copolymer described herein). The synthetic copolymer has an LCST below physiologic temperatures so that it is a solid at physiologic temperatures and dissolves at lower temperatures, such as less than 35° C. and preferably less than 30° C., 27° C. or 25° C. According to one embodiment, the copolymer comprises an N-isopropylacrylamide residue (an N-isopropylacrylamide monomer incorporated into a polymer), one or both of an acrylic acid residue and a methacrylic acid residue and an acrylic residue having an amine-reactive group.

In another non-limiting embodiment, cell growth scaffold comprises a biological polymer. In certain embodiments, a biological polymer is combined with a synthetic polymer. In one non-limiting embodiment, the biological polymer is provided in the form of an extracellular matrix-derived material. Generally, any type of extracellular matrix (ECM) can be used to prepare the biological, ECM-derived polymeric component of the cell growth scaffold (for example and without limitation, see U.S. Pat. Nos. 4,902,508; 4,956,178; 5,281,422; 5,352,463; 5,372,821; 5,554,389; 5,573,784; 5,645,860; 5,711,969; 5,753,267; 5,762,966; 5,866,414; 6,099,567; 6,485,723; 6,576,265; 6,579,538; 6,696,270; 6,783,776; 6,793,939; 6,849,273; 6,852,339; 6,861,074; 6,887,495; 6,890,562; 6,890,563; 6,890,564; and 6,893,666). By “ECM-derived material” it is meant a composition that is prepared from a natural ECM or from an in vitro source wherein the ECM is produced by cultured cells and comprises one or more polymeric components (constituents) of native ECM.

According to one non-limiting example of the ECM-derived material, ECM is isolated from a vertebrate animal, for example, from a warm blooded mammalian vertebrate animal including, but not limited to, human, monkey, pig, cow, sheep, etc. The ECM may be derived from any organ or tissue, including without limitation, urinary bladder, intestine, liver, heart, esophagus, spleen, stomach and dermis. The ECM can comprise any portion or tissue obtained from an organ, including, for example and without limitation, submucosa, epithelial basement membrane, tunica propria, etc. In one non-limiting embodiment, the ECM is isolated from urinary bladder, which may or may not include the basement membrane. In another non-limiting embodiment, the ECM includes at least a portion of the basement membrane. In certain non-limiting embodiments, the material that serves as the biological component of the scaffold consists primarily (e.g., greater than 70%, 80%, or 90%) of ECM. In another non-limiting embodiment, the biodegradable elastomeric scaffold may contain at least 50% ECM, at least 60% ECM, at least 70% ECM, and at least 80% ECM. In yet another non-limiting embodiment, the biodegradable elastomeric scaffold comprises at least 10% ECM. The ECM material may or may not retain some of the cellular elements that comprised the original tissue such as capillary endothelial cells or fibrocytes. The type of ECM used in the scaffold can vary depending on the intended cell types to be recruited during wound healing or tissue regeneration, the native tissue architecture of the tissue organ to be replaced, the availability of the tissue source of ECM, or other factors that affect the quality of the final scaffold and the possibility of manufacturing the scaffold. For example and without limitation, the ECM may contain both a basement membrane surface and a non-basement membrane surface, which would be useful for promoting the reconstruction of tissue such as the urinary bladder, esophagus, or blood vessel all of which have a basement membrane and non-basement membrane component.

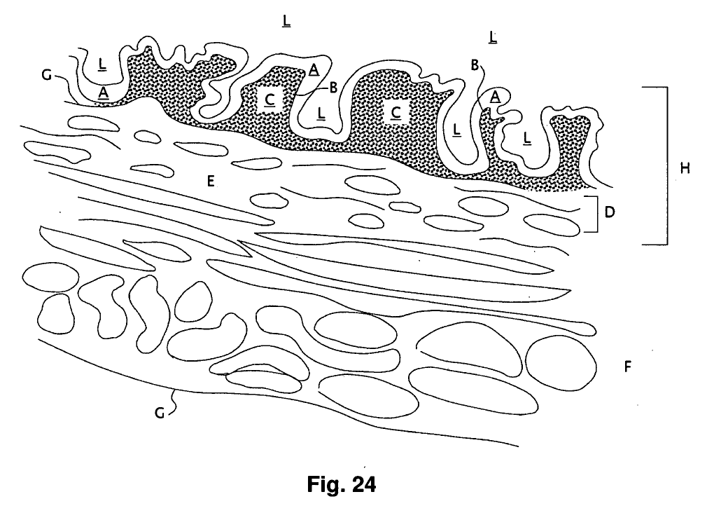

In one non-limiting embodiment, the ECM is harvested from porcine urinary bladders (also known as urinary bladder matrix or UBM). Briefly, the ECM is prepared by removing the urinary bladder tissue from a pig and trimming residual external connective tissues, including adipose tissue. All residual urine is removed by repeated washes with tap water. The tissue is delaminated by first soaking the tissue in a de-epithelializing solution, for example and without limitation, hypertonic saline (e.g., 1.0 N saline), for periods of time ranging from ten minutes to four hours. Exposure to hypertonic saline solution removes the epithelial cells from the underlying basement membrane. Optionally, a calcium chelating agent may be added to the saline solution. The tissue remaining after the initial delamination procedure includes the epithelial basement membrane and tissue layers abluminal to the epithelial basement membrane. This tissue is next subjected to further treatment to remove most of the abluminal tissues but maintain the epithelial basement membrane and the tunica propria. The outer serosal, adventitial, tunica muscularis mucosa, tunica submucosa and most of the muscularis mucosa are removed from the remaining deepithelialized tissue by mechanical abrasion or by a combination of enzymatic treatment (e.g., using trypsin or collagenase) followed by hydration, and abrasion. Mechanical removal of these tissues is accomplished by removal of mesenteric tissues with, for example and without limitation, Adson-Brown forceps and Metzenbaum scissors and wiping away the tunica muscularis and tunica submucosa using a longitudinal wiping motion with a scalpel handle or other rigid object wrapped in moistened gauze. Automated robotic procedures involving cutting blades, lasers and other methods of tissue separation are also contemplated. After these tissues are removed, the resulting ECM consists mainly of epithelial basement membrane and subjacent tunica propria (layers B and C of FIG. 24.

In another embodiment, the ECM is prepared by abrading porcine bladder tissue to remove the outer layers including both the tunica serosa and the tunica muscularis (layers G and F in FIG. 24) using a longitudinal wiping motion with a scalpel handle and moistened gauze. Following eversion of the tissue segment, the luminal portion of the tunica mucosa (layer H in FIG. 24) is delaminated from the underlying tissue using the same wiping motion. Care is taken to prevent perforation of the submucosa (layer E of FIG. 24). After these tissues are removed, the resulting ECM consists mainly of the tunica submucosa (layer E of FIG. 24).

The ECM can be sterilized by any of a number of standard methods without loss of function. For example and without limitation, the material can be sterilized by propylene oxide or ethylene oxide treatment, gamma irradiation treatment (0.05 to 4 mRad), gas plasma sterilization, peracetic acid sterilization, or electron beam treatment. Treatment with glutaraldehyde results in sterilization as well as increased cross-linking of the ECM. This treatment substantially alters the material such that it is slowly resorbed or not resorbed at all and incites a different type of host remodeling, which more closely resembles scar tissue formation or encapsulation rather than constructive remodeling. If desired, cross-linking of the protein material within the ECM can also be induced with, for example and without limitation, carbodiimide isocyanate treatments, dehydrothermal methods, and photooxidation methods. In one non-limiting embodiment, the ECM is disinfected by immersion in 0.1% (v/v) peracetic acid, 4% (v/v) ethanol, and 96% (v/v) sterile water for two hours. The ECM material is then washed twice for 15 minutes with PBS (pH=7.4) and twice for 15 minutes with deionized water. The ECM-derived material may be further processed by optionally drying, desiccation, lyophilization, freeze drying, glassification. The ECM-derived material optionally can be further digested, for example and without limitation by hydration (if dried), acidification, enzymatic digests with, for example and without limitation, trypsin or pepsin and neutralization.

Commercially available ECM preparations can also be used as the biological polymeric component of the scaffold. In one non-limiting embodiment, the ECM is derived from small intestinal submucosa or SIS. Commercially available preparations include, but are not limited to, Surgisis™, Surgisis-ES™, Stratasis™, and Stratasis-ES™ (Cook Urological Inc.; Indianapolis, Ind.) and GraftPatch™ (Organogenesis Inc.; Canton Mass.). In another non-limiting embodiment, the ECM is derived from dermis. Commercially available preparations include, but are not limited to Pelvicol™ (sold as Permacol™ in Europe; Bard, Covington, Ga.), Repliform™ (Microvasive; Boston, Mass.) and Alloderm™ (LifeCell; Branchburg, N.J.). In another embodiment, the ECM is derived from urinary bladder. Commercially available preparations include, but are not limited to UBM (Acell Corporation; Jessup, Md.). In yet another embodiment, the ECM is derived from basement membranes. Commercially available preparations include, but are not limited to BD Matrigel™ Matrix (BD Biosciences, San Jose, Calif.).

In yet another non-limiting embodiment, and as a further example of an ECM-derived cell growth scaffold, the cell growth scaffold comprises “Matrigel™,” which refers to a product provided by BD Biosciences (San Jose, Calif.). As described by BD Biosciences, BD Matrigel™ Matrix comprises solubilized basement membrane extracted from Engelbreth-Holm-Swarm (EHS) mouse sarcoma, which is a tumor that is rich in ECM proteins (see e.g. K. Ohashi, et al., M.D., Methods for Implantation of BD Matrigel™ Matrix into Mice and Tissue Fixation, BD Biosciences Technical Bulletin #455, 2006). Matrigel™ compositions comprise various components, including laminin, collagen IV, heparan sulfate proteoglycans, and entactin/nidogen. Matrigel™ compositions can also further comprises TGF-beta, epidermal growth factor, insulin-like growth factor, fibroblast growth factor, tissue plasminogen activator, and other growth factors within the EHS tumor.

Various types of Matrigel™ compositions are available, including BD Matrigel™ Matrix, BD Matrigel™ Matrix Growth Factor Reduced (GFR), BD Matrigel™ Matrix Phenol Red Free, BD Matrigel™ Matrix GFR Phenol Red Free, and BD Matrigel™ Matrix High Concentration. For example and without limitation, typical protein concentrations for BD Matrigel™ Matrix are between 9-12 mg/ml, where the typical protein concentrations for BD Matrigel™ Matrix High Concentration are between 18-22 mg/ml. In yet another non-limiting example, the BD Matrigel™ Matrix comprises 0-0.1 pg/mL of basic fibroblast growth factor, 0.5-1.3 ng/mL of epidermal growth factor, about 15.6 ng/mL of insulin-like growth factor 1, about 12 pg/mL of platelet-derived growth factor, <0.2 ng/mL of nerve growth factor, and about 2.3 ng/mL of tissue growth factor-beta, wherein about 80% of the protein gels; and BD Matrigel™ Matrix GFR Phenol Red Free comprises 0-0.1 pg/mL of basic fibroblast growth factor, <0.5 ng/mL of epidermal growth factor, about 5 ng/mL of insulin-like growth factor 1, <5 pg/mL of platelet-derived growth factor, <0.2 ng/mL of neuronal growth factor, and about 1.7 ng/mL of tissue growth factor-beta, wherein about 83% of the protein gels. These Matrigel™ compositions typically gel at about 22° C. to about 35° C.

Matrigel™ compositions can be used for various in vitro and in vivo applications, for example and without limitation, for supporting angiogenesis, cell growth, and/or implantation of tumor cells. Matrigel™ compositions can be used under several different procedures, such as by a thin gel method, a thick gel method, a thin coating method, and an injection method. The Matrigel™ compositions could optionally be diluted with a serum-free medium and/or a buffer. In one non-limiting embodiment, a Matrigel™ composition is thawed overnight at 4° C., added to a surface at 50 μL per square centimeter, and then gelled at 37° C. for 30 minutes to create a thin gel on the surface. In another non-limiting embodiment, a Matrigel™ composition is thawed, added to a surface at 150-200 μL per square centimeter, and then gelled at 37° C. for 30 minutes to create a thick gel on the surface. In yet another non-limiting embodiment, a Matrigel™ composition is thawed, diluted with serum-free media, added to a surface, gelled at about 25° C. for one hour to create a thin coating on the surface, and then rinsed with serum-free media before use. In another non-limiting embodiment, ≧10⁵ cells in media are mixed into a Matrigel™ composition on ice, and then the combination is injected into a patient using a 19 G needle for tissue samples or a 23 G needle for cultured cells.

Generally, a scaffold comprising a plurality of hollow fibers can be implanted by using any suitable medical procedure that facilitates use of the scaffold to provide a therapeutic benefit. As used herein, the terms “implanted” and “implantation” and like terms refer to an act of delivering a scaffold to a site within the patient and of affixing the scaffold to the site. The terms “implanted” and “implantation” and like terms can also refer to an act of delivering a cell growth scaffold to a site within the patient and of inserting hollow fibers through the delivered cell growth scaffold.

The site of implantation in a patient typically is “at or near a site for wound healing or tissue generation or regeneration in the patient,” meaning the scaffold-containing device is implanted in, on, onto, adjacent to or in proximity to a desired site of delivery to facilitate healing and/or tissue generation or regeneration to repair an injury or defect in the patient and/or to achieve a desired effect in the patient, such as angiogenesis or osteogenesis. The delivery method may also include minimally invasive methods such as by catheter based technology or by needle injection. The patient may be human or animal. The scaffold may be delivered by any surgical procedure, including minimally invasive techniques, such as laparoscopic surgery, as well as invasive techniques such as thoracic surgery and fasciotomy. In certain non-limiting embodiments, the scaffolds are used as surgical fabrics. For example and without limitation, the scaffolds can be implanted in a patient during laparoscopic procedures to repair or to reinforce fasciae that have been damaged or weakened. The biodegradable scaffold may be implanted alone or implanted in conjunction with surgical fasteners, such as sutures, staples, adhesives, screws, pins, and the like. Additionally, biocompatible adhesives, such as, without limitation, fibrin-based glue) may be used to fasten the elastomeric scaffolds as well.

In other non-limiting embodiments, the scaffolds may be used to promote healing of deep tissue wounds, such as puncture wounds, bullet wounds, or wounds that result from the surgical removal of a substantial amount of tissue, such as in debridement procedures or removal of tumors. A non-limiting example for implanting a scaffold would be to inject cell growth scaffold into the area of the wound and then insert hollow fibers into the cell growth scaffold. In yet another non-limiting example, a scaffold comprising a plurality of hollow fibers embedded in a cell growth scaffold could be applied into the deep tissue wound to facilitate angiogenesis.

The hollow fibers may be used for controlled delivery of one or more therapeutic agents. As used herein, the term “controlled delivery” refers to administering one or more therapeutic agents to a patient through the hollow fibers described herein. As used herein, the term “patient” refers to members of the animal kingdom including but not limited to human beings. As used herein, the terms “therapeutic agent” and “therapeutic agents” refer to any compositions having a preventative or therapeutic effect, including and without limitation, antibiotics, peptides, hormones, organic molecules, vitamins, supplements, factors, nutrients, proteins, and chemoattractants. For example and without limitation, a therapeutic agent has a therapeutic effect of stimulating angiogenesis, promoting osteogenesis, and/or reducing infection.

In certain non-limiting embodiments, the therapeutic agent is a growth factor, including cytokines, such as a neurotrophic or angiogenic factor, which optionally may be prepared using recombinant techniques. Non-limiting examples of growth factors include sphingosine-1-phosphate (S1P), bone morphogenetic proteins (BMPs, including BMP2, BMP3, and BMP5), basic fibroblast growth factor (bFGF), acidic fibroblast growth factor (aFGF), vascular endothelial growth factor (VEGF), hepatocyte growth factor (HGF), insulin-like growth factors 1 and 2 (IGF-1 and IGF-2), platelet derived growth factor (PDGF), stromal derived factor 1 alpha (SDF-1 alpha), nerve growth factor (NGF), ciliary neurotrophic factor (CNTF), neurotrophin-3, neurotrophin-4, neurotrophin-5, pleiotrophin protein (neurite growth-promoting factor 1), midkine protein (neurite growth-promoting factor 2), brain-derived neurotrophic factor (BDNF), tumor angiogenesis factor (TAF), corticotrophin releasing factor (CRF), transforming growth factors α and β (TGF-α and TGF-β), interleukin-8 (IL-8), granulocyte-macrophage colony stimulating factor (GM-CSF), interleukins, and interferons. Commercial preparations of various growth factors, including neurotrophic and angiogenic factors, are available from R & D Systems, Minneapolis, Minn.; Biovision, Inc, Mountain View, Calif.; ProSpec-Tany TechnoGene Ltd., Rehovot, Israel; and Cell Sciences®, Canton, Mass.

In certain non-limiting embodiments, the therapeutic agent is a nutrient. For example and without limitation, the nutrient promotes cell growth and/or formation of microvessels. Non-limiting examples of nutrients include amino acids, such as glycine, and glucose.

In certain non-limiting embodiments, the therapeutic agent is a coagulation factor. Non-limiting examples of coagulation factors include fibrinogen, prothrombin, tissue factor (TF), thrombin, Factor V, Factor VII, TF:VIIa complex, Factor VIII, Factor IX, Factor X, and von Willebrand factor.

In certain non-limiting embodiments, the therapeutic agent is an antimicrobial agent, such as, without limitation, isoniazid, ethambutol, pyrazinamide, streptomycin, clofazimine, rifabutin, fluoroquinolones, ofloxacin, sparfloxacin, rifampin, azithromycin, clarithromycin, dapsone, tetracycline, erythromycin, ciprofloxacin, doxycycline, ampicillin, amphotericin B, ketoconazole, fluconazole, pyrimethamine, sulfadiazine, clindamycin, lincomycin, pentamidine, atovaquone, paromomycin, diclazaril, acyclovir, trifluorouridine, foscarnet, penicillin, gentamicin, ganciclovir, iatroconazole, miconazole, Zn-pyrithione, and silver salts such as chloride, bromide, iodide and periodate.

In certain non-limiting embodiments, the therapeutic agent is an anti-inflammatory agent, such as, without limitation, an NSAID, such as salicylic acid, indomethacin, sodium indomethacin trihydrate, salicylamide, naproxen, colchicine, fenoprofen, sulindac, diflunisal, diclofenac, indoprofen, sodium salicylamide; an anti-inflammatory cytokine; an anti-inflammatory protein; a steroidal anti-inflammatory agent; or an anti-clotting agents, such as heparin. Other drugs that may promote wound healing and/or tissue regeneration may also be included.

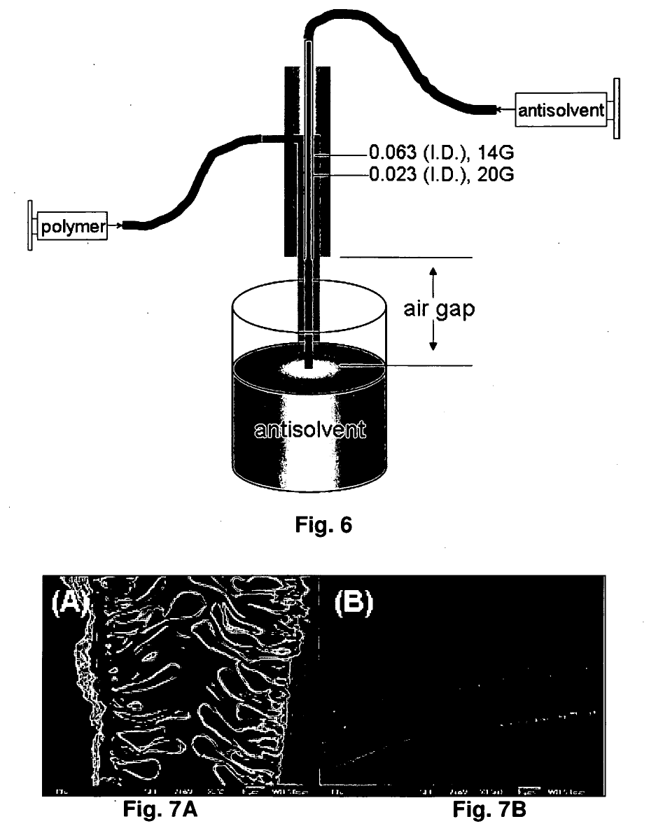

Hollow fibers may be manufactured by any suitable process, such as is shown in Example 1, below, and FIG. 6. A variety of manufacturing processes for hollow polymer tubes are known in the fabrication arts and are useful in making the hollow fibers described herein so long as the temperature of the tubing can be maintained above the LCST after formation, and conversely, below the LCST prior to hollow fiber formation. Given that these fibers are for implantation in a patient, sterility also is required. The hollow fibers can have any useful inner (lumen) and outer diameters. The thickness of the filament walls (outer diameter minus inner diameter divided by 2) should be sufficient to provide sufficient strength to the structure and to prevent premature erosion of the hollow filaments due to the degradation process. The inner diameter should be sufficiently large to permit passage of any material, such as cell- or growth factor-containing compositions, through the hollow filament. As such, the inner diameter of the hollow filaments might range, without limitation from 0.1 inches (the nominal inner diameter of a 10 gauge needle) to 0.0035 inches (the nominal inner diameter of a 35 gauge needle). The minimum inner diameter of the hollow filaments may be less than 0.0035 inches, so long as the filaments remain useful for transfer of compositions. It should be recognized that the inner diameter may expand under sufficient pressure due to the elasticity of the copolymers or other materials described herein, and, as such, very small inner diameters may be used. The inner and outer diameters may vary from one end or portion of the hollow fiber to another, for example, with a thinner end for implantation and a thicker end to extend outside the body of a patient for connection to a pump or syringe.

Compositions comprising a copolymer described herein can be distributed for use in any suitable vessel. In one instance, the composition is packaged in a sealed container, from which the composition can be removed. The composition may be transported within a liquid in a pouch. The liquid would help protect the structure and would retain heat, assisting in maintaining the temperature of the structure above the LCST of the copolymer from which the structure is made.

In one non-limiting embodiment, the hollow fiber is manufactured using an injection process with a double concentric nozzle, which accommodates a smaller diameter tubing within a larger diameter tubing. A solution containing the polymer is injected into the larger diameter tubing, whereas the antisolvent is injected into the smaller diameter tubing. The space between the distal end of the double concentric nozzle and a receptacle to collect the fibers is called the “air gap.” Starting from the air gap and within the receptacle, the polymer precipitates to form the hollow fibers.

The hollow fibers can be characterized by various physical and chemical characteristics, such as elastic modulus, tensile strength, LCST, and void fraction. As used herein, “void fraction” refers to the ratio of the volume taken up by the void as compared to the total volume of the material. A variety of analytical processes for measuring void volumes are known in the technical arts. For example and without limitation, void fraction can be determined by image analysis of a photomicrograph of a cross section of a hollow fiber, wherein the area of the image taken up by the void is compared to the total area of the image.

In a further embodiment, a cell culture apparatus is provided, comprising a culture vessel containing any embodiment of the hollow fibers described herein. The culture vessel may be any useful configuration, including flasks, bottles, plates, cylinders, etc. and can include a mechanism for culture medium replenishment, such as continuous or substantially continuous replenishment of culture medium, as is understood in the context of open culture systems. The hollow fibers would extend from within the culture vessel or be fluidly connected to an external pump or other “feed” outside the culture vessel, such as a syringe or IV bag. As used herein, the term “fluid connector” and like terms refer to one or more structures that facilitates extension of a fluid path, for instance and without limitation, from a hollow fiber within a culture vessel to outside the culture vessel. Non-limiting examples of fluid connectors include valves, tubing, Y-junctions, reservoirs, channels, and tubing connectors.

The hollow fibers can be incorporated into a cell growth scaffold, as described above.

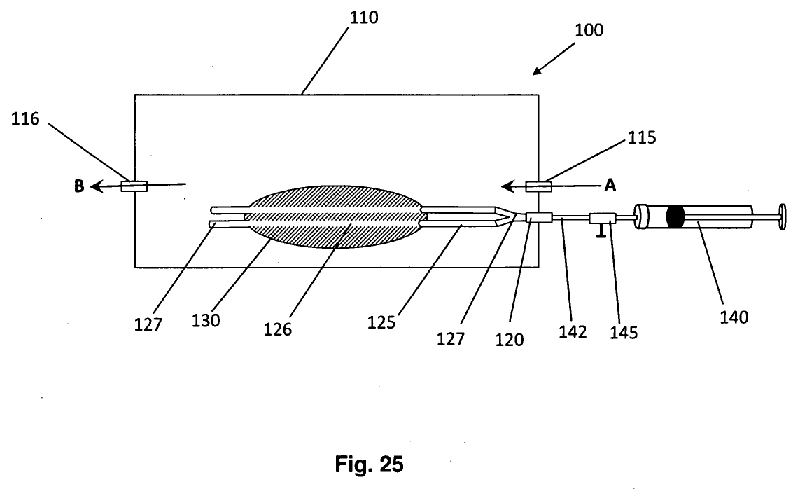

FIG. 25 shows one embodiment of this cell culture apparatus 100 shown schematically to illustrate the general set up of one type of such as system. The cell culture apparatus 100 comprises a culture vessel 110. Cell culture apparatus 100 is an open system, meaning that culture media can continuously flow into culture vessel 110 via inlet 115, depicted by arrow A, and out of culture vessel 110 via outlet 116, depicted by arrow B (medium supply container, connected to inlet 115 and medium waste container, connected to outlet 116 are not shown). Connector 120 is used to fluidly connect hollow fibers internal to the culture vessel 110 with an external fluid source. Depicted are hollow fibers 125 and 126, as described herein, attached to connector 120 via a Y-junction 127. Hollow fibers are partially embedded in a cell culture scaffold 130, which may be configured into any desired shape, such as the shape of joint, ear or nose cartilage, or a bone structure. Portions of the hollow fibers 126 are embedded in scaffold 130, while other portions of the hollow fibers 125 protrudes from and are external to the scaffold 130. As is shown in FIG. 25 and as is applicable, without limitation, to all embodiments in which hollow fibers are embedded in a cell growth scaffold, a first portion of the hollow fibers (e.g., fibers 126) are embedded within the scaffold 130, while a second portion 125 protrudes from (is external from) the scaffold 130 and a third portion 127 protrudes from scaffold 130 opposite the second portion 125. A syringe 140 is shown attached to connector 120 via a tube 142. A valve 145 is shown in-line between the syringe 140 and connector 120 in tube 142 to permit changing of the syringe without backflow from the hollow fibers 125 and 126. Syringe 140 is used to introduce nutrients, growth factors, cytokines, drugs, etc. into the scaffold to promote growth and/or differentiation of cells within the matrix. It will be appreciated by those of ordinary skill in the art, that the size, shape and configuration of the cell culture apparatus may be varied for any reason whatsoever and will achieve equivalent results. Additional features, such as a gas inlet, circulation impellers, baffles, supports, etc. may be added as a matter of design optimization, typically for a particular use.

In use, the scaffold 130 and hollow fibers 125 and 126 are placed in vessel 110 and are connected to connector 120. Scaffold 130 may be pre-seeded with cells. Culture medium can then be introduced into the vessel 110 via inlet 115 and exits through outlet 116. Culture medium typically is constantly introduced into vessel 110 to promote optimal nutrient support. Nevertheless, culture medium may be introduced at regular or irregular intervals. Inlet 115 and outlet 116 may be omitted in favor of a simple opening with any useful closure means, such as in a cell culture flask. Connector 120 may pass through closure means, such as a screw top. Vessel 110 may be rocked, shaken or rolled, to agitate the medium, or the medium otherwise can be agitated by stirring or any other useful means. Cells, nutrients, cytokines, growth factors, antibiotics, therapeutic agents, etc. can be introduced into the hollow fibers 125 and 126 by using a syringe as shown in FIG. 25, by a drip/gravity feed method such as by use of an IV bag, or by a pump, such as a peristaltic pump. In use, nutrients, cells, cytokines, etc. may be fed into the hollow filaments over any desirable temporal period to facilitate growth of tissue. As would be apparent, the cell growth scaffold may be tailored specifically in shape or size to any tissue that is to be grown. For example, cartilage tissue may be produced by use of molded structures in, e.g., ear, nose, or knee cartilage shapes, which may be customized for any individual. Autologous chondrocytes may be seeded into the matrix via the hollow filaments. Appropriate nutrients and cytokines may be fed in any suitable temporal profile via the hollow filaments. The pre-seeded, and pre-cultured cell-growth matrix, including the hollow filaments, may be transplanted into a patient at any suitable time, and additional nutrients or cytokines, including angiogenic factors, can be fed to the cell growth scaffold via the hollow filaments. Once the graft is established, the hollow fibers can be dissolved as described herein.

In a related embodiment, also provided is a method of culturing tissue in vitro. The method comprises culturing tissue in a cell culture apparatus as described above.

The following examples are provided for illustration purposes and are not intended to limit the scope of the present invention.

### EXAMPLE 1

### Fabrication and Characterization of Hollow Fibers for Controlled Delivery

We have fabricated porous hollow fibers made from the model, triggerably-dissolvable material, cellulose, and our new synthetic, temperature-sensitive elastomer. The pore size and structure of these hollow fibers is dependant upon the fabrication conditions. Using scanning electron microscopy (SEM), we have characterized these fibers by examining fiber void fraction. We have also shown that these fibers can be used to controllably release small molecules and proteins from the lumen of these fibers to the surrounding environment. This release rate is dependant on the porosity of the fiber.

Additionally, we used single hollow fibers to controllably release an angiogenic growth factor (sphingosine-1-phosphate, S1P) from the lumen of a hollow fiber in vitro. A concentrated solution of this growth factor was injected into the lumen of a single hollow fiber embedded in an extracellular matrix (ECM)-mimetic material (Matrigel™) in order to verify biological activity through a tubular formation assay. We have shown that when S1P is injected into hollow fibers embedded in Matrigel™ and human umbilical vein endothelial cells (HUVECs) are seeded onto the Matrigel™, the endothelial cells form tubular structures. The area of Matrigel™ covered by these tubular structures is dependant on the concentration of S1P injected into the fiber and thus, the concentration of S1P released to the cells. This demonstrates that we have external control over the behavior of endothelial cells in extracellular matrix.

Inducing angiogenesis has been a major focus of medical and tissue engineering research as it has the potential to facilitate wound healing in deep wounds where the vasculature greatly damaged. Research in this field has focused on controlled release of growth factors to promote or speed up native vasculature recruitment. Studies have shown that it is possible to controllably release active growth factors from a scaffold both in vitro (Kawakami O, et al., Neurosurgery, 2006. 58(2): 355-64; Lazzeri L, et al., Polymer International, 2005. 54: 101-107; Wacker B K, et al., Delivery of sphingosine 1-phosphate from poly(ethylene glycol) hydrogels. Biomacromolecules, 2006. 7(4): 1335-43) and in vivo (Tilakaratne H K, et al., Biomaterials, 2007. 28(1): 89-98; Tabata Y, et al., Controlled release of vascular endothelial growth factor by use of collagen hydrogels. J Biomat Sci Polym ed, 2000. 11(9): 915-30). Recent work involving the release of VEGF from a polymeric scaffold serves as proof in principle for the benefit of controlled growth factor release in tissue engineering (Ennett A B, et al. J Biomed Mat Res A, 2006. 79: 176-84). However, healing is a complex, multistage, cascade of events and would require a multifaceted schedule of many growth factors and nutrients. Release of such factors from a scaffold would require programming this complex release schedule prior to implantation. Unfortunately, the sheer number of factors required in different amounts, at different stages and over extended timeframes, makes such a task daunting if not unfeasible using current controlled release technology. Furthermore, it is unlikely that a given formulation would be suitable for a diverse population of individuals and the associated variability in the healing for each wound site and patient. Unfortunately, the number of factors required in different amounts, at different stages and over extended timeframes, makes such a task unfeasible using current controlled release technology. Furthermore, it is unlikely that a given formulation would uniformly suit different individuals let alone the associated variability in healing different tissues.

A completely new approach to solving this problem of complex release from a scaffold would be to incorporate externally-regulated “synthetic vasculature” into a wound site where the native vasculature is damaged. Porous, hollow fibers could work in a similar fashion to native vasculature to deliver appropriate nutrients and/or growth factors. However, instead of a closed system, feed and waste removal would be maintained externally so that supply to the new tissue could be provided on an as-needed basis. Additionally, this would serve as a modular delivery strategy that is adaptable to a patient's individual need.

For this technology to be realized, a method of removing the device following wound healing must be devised. This synthetic capillary, which would be embedded in neo-tissue, cannot be forcibly removed, as it will create unwanted damage. Instead, the material should be able to be dissolved or degraded by triggering a specific pathway, which can be enzymatic-, temperature-, or pH-based. In the case of an enzymatic trigger, it is important that the enzyme that breaks down the synthetic capillary system is not capable of damaging the newly formed tissue. Cellulose is a potential candidate material for such a device because naturally derived-cellulose has been used as a biomaterial for various applications, such as hollow fibers, because the enzyme cellulase specifically degrades cellulose without damaging neo-ECM (Ko I K and Iwata H, An approach to constructing three-dimensional tissue. Ann NY Acad. Sci., 2001. 944: 443-55).

In the laboratory setting, porous hollow fibers can be fabricated from cellulose to release various sized agents through controlling the size of pore and macrovoids which result from spinodal decomposition (van de Witte P, et al., Phase separation processes in polymer solutions in relation to membrane formation. J Memb Sci, 1996. 117: 1-31). We demonstrate control over the properties of fibers, including pore size, in the following section. However, delivery of large proteins (angiogenesis promoters can be as large as 46 kD in their dimeric, active form) may necessitate relatively large pore sizes, and thus, greater mechanical stability of the surrounding material than if the pores were smaller. Synthetic membranes can also be utilized, provided a material-specific technique for a triggered degradation exists. Materials like poly(N-isopropylacrylamide) (NIPAM), demonstrate a temperature induced dissolution effect over a biologically-relevant temperature range (from 37° C. to 32° C.) (Zhang J and Peppas N A, Synthesis and Characterization of pH- and Temperature-Sensitive poly(methacrylic acid)/Poly(N-isopropylacrylamide). Macromolecules, 2000. 33: 102-107; Kono K, et al. Effect of poly(ethylene glycol) grafts on temperature-sensitivity of thermosensitive polymer-modified liposomes. J Controlled Release, 2002. 80: 321-32). However, conventional NIPAM has considerably poor mechanical stability and also does not have a sharp phase transition. When small modifications, such as the molar ratio of methacryloxypropyltrimethoxysilane (MPS, a co-polymer) to NIPAM are made, the elastic modulus increases from 50 kPa to almost 200 kPa (Ho E Y, Engineering Bioactive Polymers for the Next Generation of Bone Repair. 2005, Drexel University). Recently, we have synthesized an elastomeric polyurethane copolymer including NIPAM that has proven to be remarkably tough at body temperature while also exhibiting an extremely sharp transition to solubility in water at room temperature. Previously, NIPAM-based polymers have been used both as a scaffold for tissue engineering (Au A, et al., Thermally reversible polymer gel for chondrocyte culture. J Biomed Mat Res A, 2003. 67(4): 1310-9; Naito H, et al., Three-dimensional cardiac tissue engineering using a thermoresponsive artificial extracellular matrix. American Society for Artificial Internal Organs Journal, 2004. 50(4): 344-8) and as a major component in several drug release devices (Mathews A S, et al., Drug delivery system based on covalently bonded poly[N-Isopropylacrylamide-co-2-Hydroxyethylacrylate]-based nanoparticle networks. Drug Delivery, 2006. 13: 245-251). This polymer has excellent mechanical stability/elasticity at body temperature and undergoes a sharp phase transition to solubility at reduced temperatures. We propose that this new temperature-sensitive polymer can be used to fabricate hollow fibers as an alternative to cellulose.

Fibers made from either of our two candidate materials have the potential to deliver oxygen, nutrition and even growth factors to developing tissue. Once new tissue is formed, the inlet flow could be altered to trigger dissolution of the fiber (cellulase in the case of cellulose and a lower temperature saline in the case of the new NIPAM based synthetic material), washing away the inner layer and breaking it down into small degradable pieces, which would eventually be removed by the reticuloendothelial system (RES). We hypothesized that that porous hollow fibers fabricated from such materials would serve as an externally-regulated delivery system that can be triggerably dissolved through a simple change in the properties of the feed. Success of such technology has tremendous potential for use in tissue regeneration/wound healing.

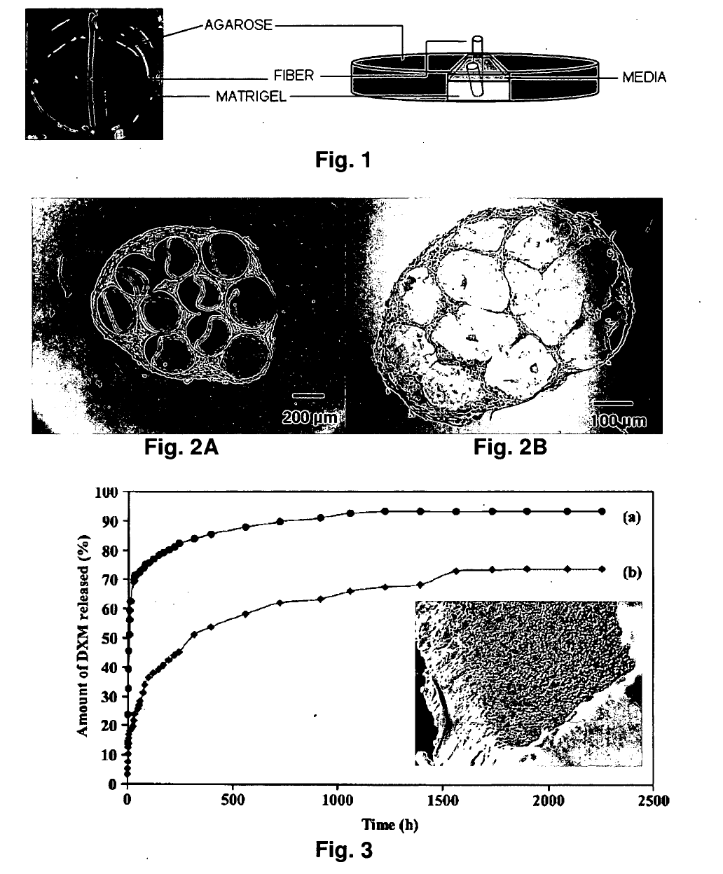

In order to reduce this technology to practice, we have constructed an in vitro system involving the delivery of sphingosine-1-phosphate (S1P), a lipid that has shown to enhance angiogenesis (Wacker B K, et al., Biomacromolecules, 2006. 7(4): 1335-43), human umbilical vein endothelial cells (HUVECs) and Matrigel™, widely used extracellular matrix mimetic material that is composed mostly of basement membrane proteins. This in vitro system (FIG. 1) allows us to deliver angiogenic growth factors to HUVECs so that we can measure the angiogenic capabilities of these cells in a biomimetic environment.

### Methods and Results

Cellulose Hollow Fiber-Based Scaffolds for Tissue Engineering. Although, to our knowledge, cellulose fibers have thus far not been used in controlled delivery for wound healing, researchers are attempting to use cellulose hollow fibers as a substrate for tissue engineering. Bundles of hollow fibers cultured with Bovine Coronary Artery Smooth Muscle Cells have been explored by Ko et al. as a substrate to grow three-dimensional tissue with a built in capillary system in vitro (Ko I K and Iwata H. Ann NY Acad Sci, 2001. 944: 443-55).

After 40 days in culture, the cells bound together to inhabit the space surrounding these bundles (FIG. 2A). Cellulase was used to dissolve the cellulose scaffold, leaving behind three dimensional tissue with a capillary-like morphology (FIG. 2B).

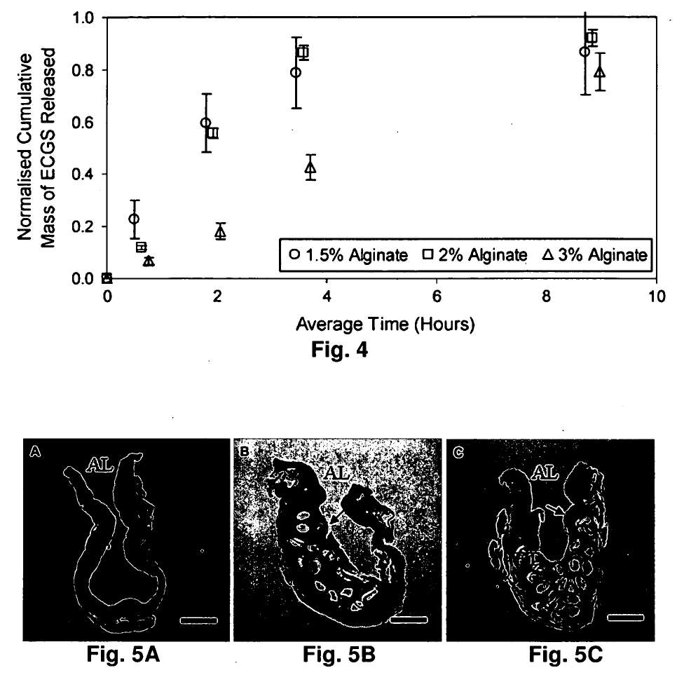

Controlled Release from Hollow Fibers. It has been demonstrated that proteins and small molecules can be released from porous hollow fibers in both an in vitro and in vivo setting. Lazzeri (Lazzeri L, et al., Polymer International, 2005. 54: 101-107) showed that the lumen of PLLA hollow fibers can be coated with PLGA microspheres (loaded with dextromethasone, DXM) during the fiber fabrication process (FIG. 3, inset). In an in vitro setting, the drug release of DXM from these hollow fibers was comparable to the drug release from the microspheres alone (FIG. 3). Additionally, Tilakaratne (Tilakaratne H K, et al. Biomaterials, 2007. 28(1): 89-98) was able to show release of endothelial cell growth supplement (ECGS) from an alginate gel filling the lumen of polypropylene hollow fibers. In this example, the alginate gel concentration was responsible for the release rate of ECGS (FIG. 4). Lastly, Kawakami (Kawakami O, et al., Neurosurgery, 2006. 58(2): 355-64) was able to use a polyethylene hollow fiber to release basic fibroblast growth factor (bFGF) in order to facilitate fibrous tissue formation in a surgically created aneurysm in a rabbit model. In this study, bFGF was loaded into a collagen gel in the lumen of a polyethylene hollow fiber. Implantation into the wound site showed that bFGF was released and remained functional as it was able to guide the aneurysm to create fibrous tissue (FIG. 5C) better than the untreated aneurysm (FIG. 5A) and an empty hollow fiber (FIG. 5B) after three weeks.

NIPAM-based Elastomer Characteristics. An elastomer was synthesized from n-isopropylacrylamide (NIPAM), n-acryloxysuccinimide, acrylic acid and a macromer hydroxyethyl methacrylate-oligomer polylactide using free radical polymerization, having the specific composition P(NIPAAm-co-AAc-co-NHS-co-HEMAPLA2.1) 85/6/4/4. Table 6 provides exemplary mechanical properties of NIPAM copolymers. Copolymers were manufactured in the manner described above. The resulting polymer has a lower critical solution temperature of 26.0° C. before degradation, a tensile strength of 0.6 MPa and 1400% elongation at failure. As opposed to conventional NIPAM, the properties of this new material are superior for fabrication of hollow fibers which would greatly benefit from the added strength/elasticity.

TABLE 6
NIPAM-based polymers and their mechanical properties

| | Conventional | New NIPAM- |
|---|---|---|
| | NIPAM | based copolymer |
| Elastic modulus | 50 kPa | 0.6 mPa |
| Lower Critical Solution | 32° C. | 26.0° C. |
| Temperature | | |

Hollow Fiber Fabrication. We have created a hollow fiber fabrication system consisting of two syringe pumps (Multi-Phaser™ NE-1000), a concentric double injection nozzle and solvent-resistant perfluoroalkoxy (PFA) tubing. The tubing connects the syringes loaded into the pumps to the nozzle (FIG. 6). The antisolvent for a specific polymer flows through the 20 G tube and the polymer flows through the 14 G tube. Both the antisolvent and polymer are extruded into a bath of antisolvent, precipitating the polymer and forming a hollow fiber.

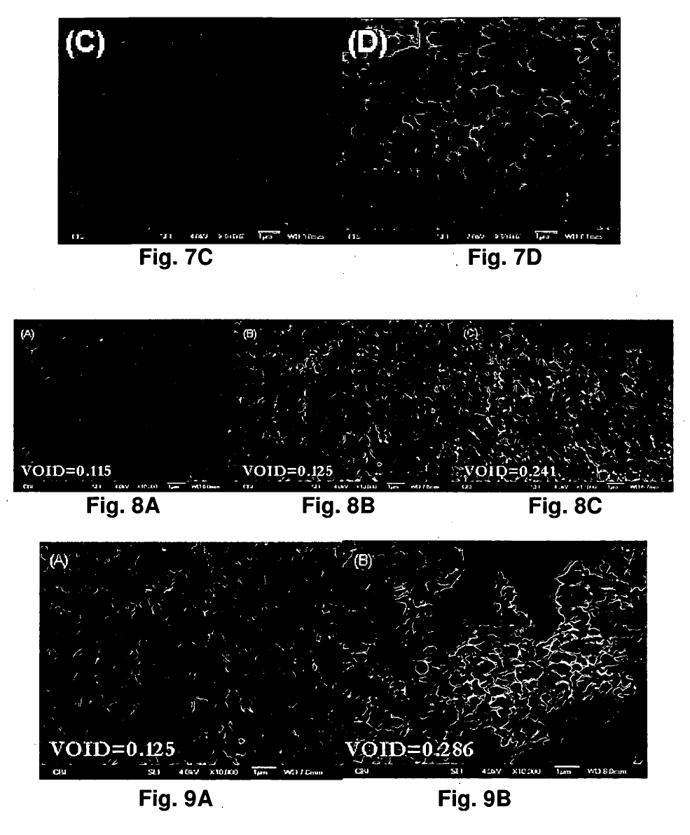

Using this setup we have fabricated hollow fibers from both cellulose acetate and a NIPAM-based, synthetic elastomer (FIG. 7). Recently, an elastomeric polyurethane copolymer was synthesized from n-isopropylacrylamide (NIPAM), n-acryloxysuccinimide, acrylic acid and a macromer hydroxyethyl methacrylate-oligomer polylactide using free radical polymerization. This material has proven to be remarkably tough at body temperature, while also exhibiting an extremely sharp transition to solubility in water at room temperature. Hydrogels of this material exhibit elongation higher than 1330% and tensile strength greater than 0.6 mPa (Guan J, et al. Protein-reactive, thermoresponsive copolymers with high flexibility and biodegradability. Biomacromolecules. 2008 April; 9(4): 1283-92). Previously, NIPAM-based polymers have been used both as a scaffold for tissue engineering (Au A, et al. Thermally reversible polymer gel for chondrocyte culture. J Biomed Mat Res A 2003; 67(4):1310-19 and Naito H, et al. Three-dimensional cardiac tissue engineering using a thermoresponsive artificial extracellular matrix. Am Soc Artif Int Org J 2004; 50(4):344-48) and as a major component in several drug release devices (Mathews A S, et al. Drug delivery system based on covalently bonded poly[N-Isopropylacrylamide-co-2-Hydroxyethylacrylate]-based nanoparticle networks. Drug Delivery 2006; 13:245-51). The copolymers described herein exhibit excellent mechanical stability/elasticity at body temperature and undergoes a sharp phase transition to solubility at reduced temperatures. Additionally, the degradation products of this material has demonstrated a lack of cytotoxicity when exposed to smooth muscle cells in culture (Guan J, et al. Biomacromolecules. 2008 April; 9(4): 1283-92). We show herein that the described temperature-sensitive copolymers can be used to fabricate hollow fibers.

The polymer solution conditions and fabrication conditions are provided in Table 7. Water was used as the antisolvent for cellulose and hexanes were used as the antisolvent for the NIPAM-based elastomer. Cross sections of the walls of the two types of hollow fibers, as well as the pore microstructure can be seen in the SEM micrographs in FIGS. 7A-7D. Fibers were lyophilized and mounted on SEM stubs to expose the cross section. The samples were sputter coated with 3.5 nm of gold-palladium and images were taken using a JEOL 9335 SEM at 5 kV.

Cross sections of the walls of the two types of hollow fibers, as well as the pore microstructure can be seen in the SEM micrographs in FIG. 7. The cellulose fiber shows large finger-like macrovoids caused by quick demixing of the solvent and antisolvent (FIG. 7A). In addition, the microporous structure of the cellulose fiber is jagged (FIG. 7C). The NIPAM-based fibers show very few spherical macrovoids (1-2 μm) at the wall's center indicating spinodal decomposition (FIG. 7B) and spherical micropores similar in size to those of the cellulose fibers (FIG. 7D). However, the micropores of the NIPAM-based hollow fibers are more consistent in shape, likely because this material is synthetic, whereas the cellulose material is naturally occurring.

TABLE 7
Polymer solution components by weight

| | | | | Polymer (wt %) | |
|---|---|---|---|---|---|
| | Solvent (wt %) | | | Cellulose | |
| | Acetone | DMSO | Isopropanol | Acetate | Elastomer |
| Cellulose | 15 | 50 | 15 | 20 | 0 |
| fiber | | | | | |
| NIPAM-based | 70 | 0 | 0 | 0 | 30 |
| fiber | | | | | |

Factors such as: 1) polymer solution concentration, 2) polymer solution and antisolvent flow rates, 3) amount of antisolvent in the polymer, and 4) amount of solvent in the antisolvent bath, all affect pore formation in membranes by altering solvent/polymer demixing and ultimately the microstructure of the fiber (van de Witte P, et al., J Memb Sci., 1996. 117: 1-31; Nunes S P and Inoue T, Evidence for spinodal decomposition and nucleation and growth mechanisms during membrane formation. J Memb Sci., 1996. 111:93-103; Shih C H, et al., Morphology of membranes formed by the isothermal precipitation of polyamide solutions from water/formic acid systems. J Appl Polym Sci., 2005. 96: 944-60). Consequently, this microstructure, consisting of pores within the polymer, determines the release capabilities of the fiber (Hasegawa M, et al., High release of antibiotic from a novel hydroxyapatite with bimodal pore size distribution. J Biomat Res B, 2004. 70(2): 332-9). By fabricating cellulose acetate hollow fibers, with varying water flow rates (10 mL/min-20 mL/min) and cellulose flow rates (1.5 mL/min-2.0 mL/min), we were able to determine how these factors affect pore size and void fraction. Qualitatively, an increase in the water flow rate increases both pore size and void fraction, and an increase in the polymer flow rate increases void fraction (FIGS. 8, 9). Void fraction was measured using MetaMorph® (Molecular Devices™) threshold analysis and void fraction was used to select representative fibers in the controlled release experiments (following section).

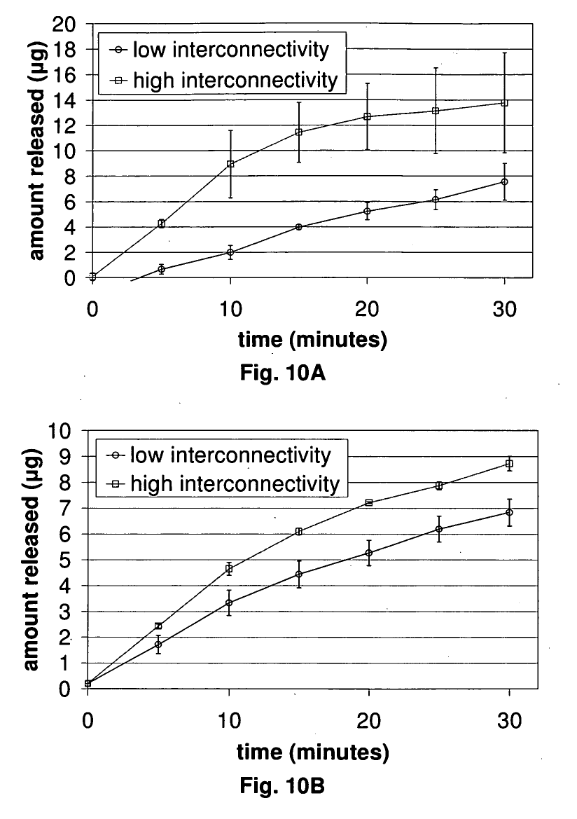

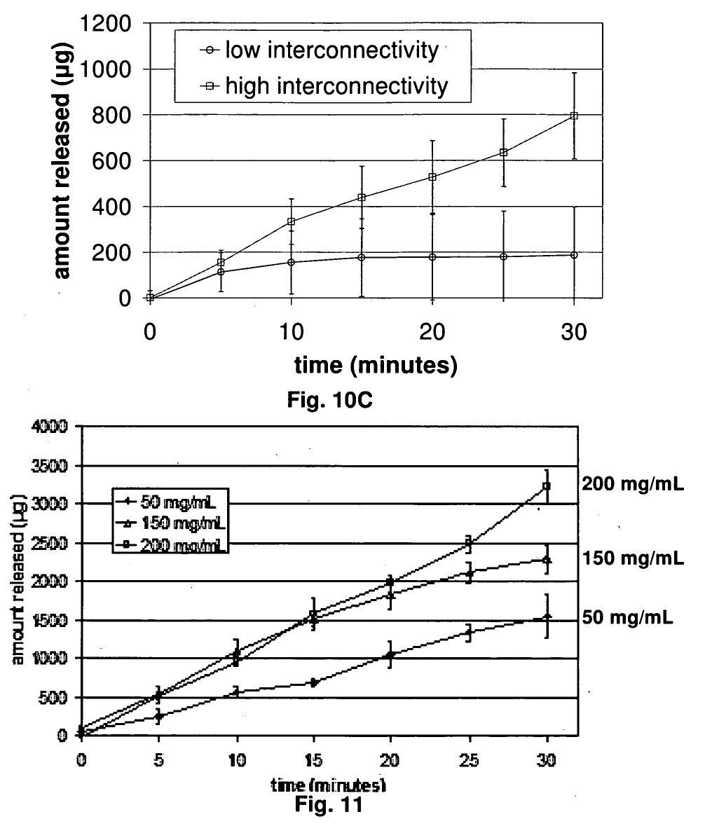

Controlled Release from Cellulose Hollow Fibers. Molecules of known sizes spanning a wide range of molecular weights (fluorescein MW=330 Da, cytochrome c MW=12 kDa, and bovine serum albumin (BSA) MW=66 kDa) were released from the lumen of fibers chosen to represent conditions with low interconnectivity of pores (FIG. 8A) and high interconnectivity of pores (FIG. 9B). This interconnectivity is related to the measured void fraction of the fiber membrane, with a higher void fraction allowing for transport at a higher rate. Using a syringe, concentrated solutions of fluorescein (50 μL, 200 μg/mL), cytochrome c (50 μL, 5 mg/mL), and BSA (50 μL, 50 mg/mL) were injected into the lumen of single hollow fibers. The fibers were immersed in 5 mL of phosphate buffered saline (PBS), and after 5 minutes, rinsed and placed in a fresh bath of 5 mL PBS. This process was repeated a total of 4 times for a duration of 30 minutes. Release of fluorescein, cytochrome c, and BSA was measured by sampling the spectra of supernatants using a SpectraMax M5 plate reader (Molecular Devices™) as illustrated in FIG. 10. Note that the total amount released in each case (y-axis) does not permit direct comparison between the plots because different concentrations of each molecule were loaded into the lumen of the fibers. This was done in order to release the amount required for detection in the linear range of our plate reader, consequently demonstrating our control over the amount released through the contents of the lumen. Instead, the important comparison in each plot is the relative amount of an individual factor released from the two different fibers where loading was equivalent (line for “low interconnectivity” versus line for “high interconnectivity” in the same graph).

Fluorescein (λₑₓ=490 nm and λₑₘ=520 nm), cytochrome c (λ_{ab}=410 nm) and BSA (λ_{ab}=280 m) release data shows that fibers with high interconnectivity (as determined by SEM) are able to release molecules or proteins in greater quantities than fibers with low interconnectivity. This is more apparent with higher molecular weight molecules (FIGS. 10B and 10C) when compared to smaller molecules (FIG. 10A). Also, BSA is below the molecular weight cutoff (MWCO) for the high interconnectivity fiber (square symbols in FIG. 10C) but above the MWCO for the low interconnectivity fiber as indicated by the elimination of BSA release after 20 minutes (circle symbols in FIG. 10C). The MWCO represents the largest sized molecule that can continually diffuse through a given pore network (from the lumen to the surface of the fiber) in the presence of a gradient. The release of BSA from a fiber with high interconnectivity was compared when injected into the lumen at varying concentrations (50 mg/mL, 150 mg/mL, 200 mg/mL). FIG. 11 illustrates how an increase in the concentration in the lumen of the fiber leads to a corresponding increase in the rate of release. These experiments demonstrate our control over the release for a range of factors of porous hollow fibers composed of cellulose.

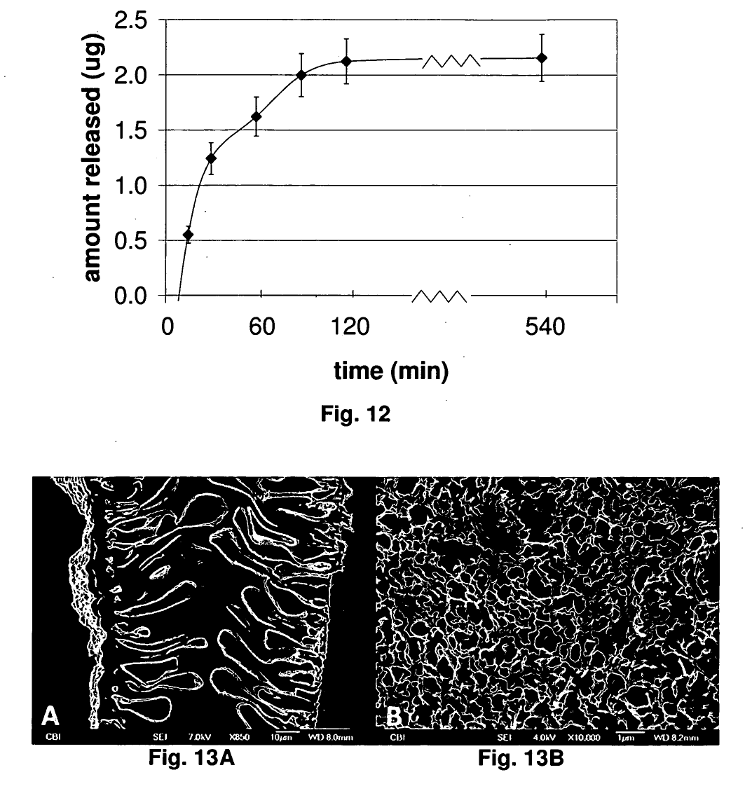

In vitro Release Setup. 2% agarose gels were cast in 6-well plates, and a 1 cm wide strip was removed from the center axis to create a well that was filled with a hollow fiber and the commercially available matrix, Matrigel™ (FIG. 1). Matrigel™ is rich in proteins found in extracellular basement membrane in vivo, making it amenable to endothelial cell attachment, function and migration. This experimental setup allows us to culture cells on top of Matrigel™ while releasing factors and maintaining a gradient originating from the fiber. Agarose is used so that a minimal amount of Matrigel™ can be applied to a larger well, facilitating the incorporation of a fiber. 50 μL of fluorescein (200 ug/mL, MW=330) was injected into the lumen of the fiber and 1 mL of media (M199) was added on top of the gels. Gels were incubated at 37° C. and fluorescein release was measured by sampling the supernatant and measuring fluorescence emissions over 9 hours. Preliminary results demonstrate that fluorescein can be released from the fiber and through the Matrigel™ for approximately 1.5 hours in a reasonably linear fashion (FIG. 12). After this time, the release rate drops and levels off to zero for the remainder of the assay.

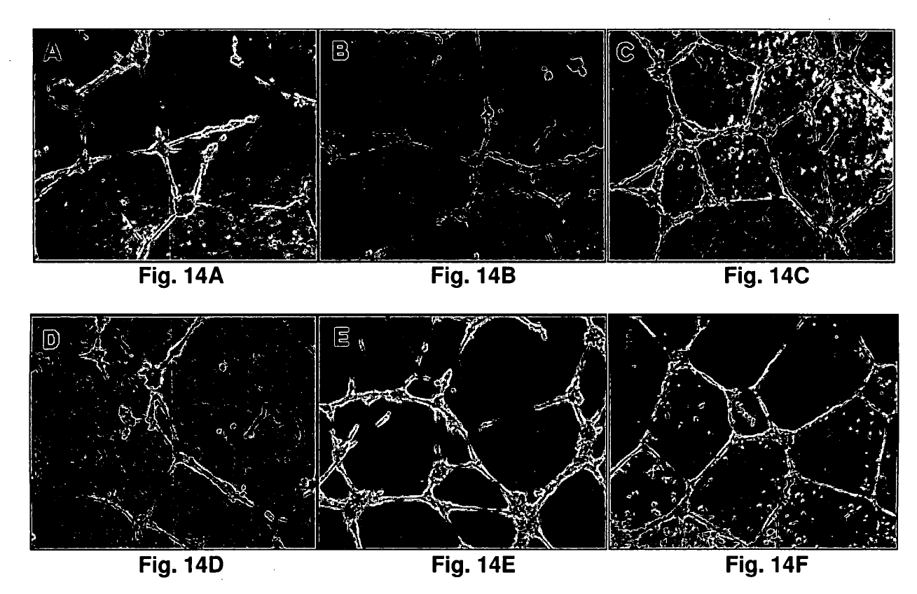

Cellular Response to Controlled Release. The bioactivity of released S1P was verified by culturing HUVECs on Matrigel™ in which a single hollow fiber has been embedded and loaded with S1P (FIG. 1). Cells did not migrate into the adjacent 2% agarose matrix given that resulting pore sizes are typically less than 2 nm (Narayanan J, et al. Determination of agarose gel pore size: Absorbance measurements vis a vis other techniques. Journal of Physics: Conference Series 2006. 28: 83-86). Cells were seeded on Matrigel™ (300,000 cells/well) with 1% FBS and M199 media containing S1P (600 mM-1800 mM) was injected into the hollow fibers. Note that S1P (Wacker B K, et al., Biomacromolecules, 2006. 7(4): 1335-43) have been previously demonstrated to exhibit sustained release from a hydrogel (similar to Matrigel™) over this same period of time. After 16 hours of culture, cells were imaged via phase/contrast microscopy to look for tubular formation. Tubular formation was assessed by measuring the area of the gel covered by capillaries using threshold analysis with MetaMorph®. Endothelial cell migration into extracellular matrix gels has been previously observed in vitro (Rajashekhar G, et al., Continuous endothelial cell activation increases angiogenesis: Evidence for the direct role of endothelium linking angiogenesis and inflammation. J Vasc Res., 2006. 43: 193-204; Takei T, et al., Fabrication of endothelialized tube in collagen gel as starting point for self-developing capillary-like network to construct three-dimensional organs in vitro. Biotechnology and Bioengineering, 2006. 95: 1-7) as well as in vivo (Wacker B K, et al., Biomacromolecules, 2006. 7(4): 1335-43). The experimental groups in this study are (1) Negative Controls: no fiber (FIG. 14A) and fiber embedded in Matrigel™, injected with M199 media (FIG. 14B), (2) Fiber embedded in Matrigel™, injected with S1P solution (600 mM-1800 mM, FIGS. 14D-F), and (3) Positive Control: no fiber, Matrigel™, media with S1P (5 μM, FIG. 14C).

FIG. 14 shows the phase/contrast images of the HUVECs seeded on Matrigel™. When HUVECs are not exposed to S1P (FIGS. 14A and 14B), little tubular formation is observed, as expected. When 5 μM S1P is added to the media, tubular formation can be observed (FIG. 14C). When highly concentrated S1P is loaded into the embedded hollow fiber, tubular formation is observed in increasing amounts with increasing S1P loading concentrations (FIGS. 14D-F). When the S1P lumen concentration is 600 mM, scattered tubular structures can be observed (FIG. 14D). When the S1P lumen concentration is 1200 mM, tubular structures can be observed, but not always complete and connected to another structure (FIG. 14E). When the S1P lumen concentration is 1800 mM, complete tubular structures can be observed (FIG. 14F).

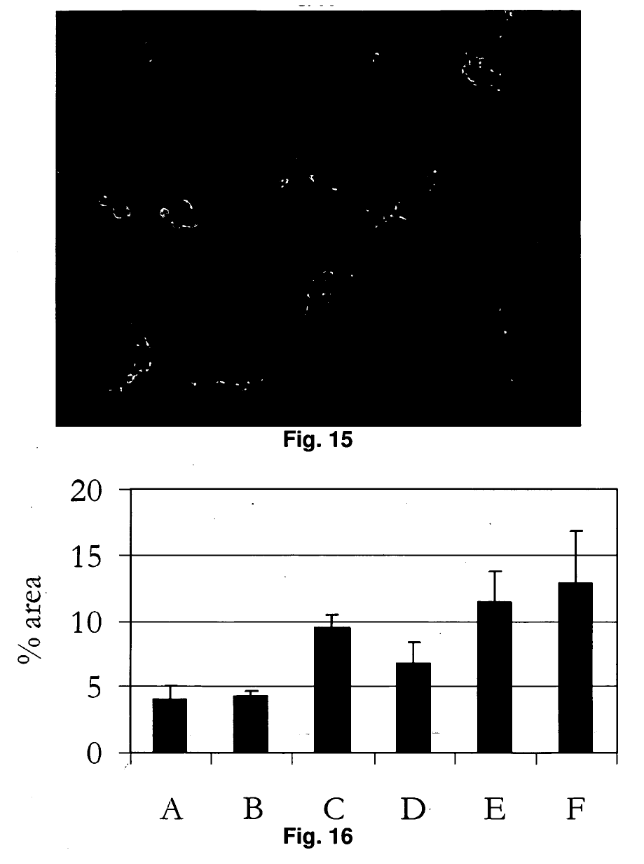

In order to quantify tubular formation, cells were fluorescently stained for F-actin (FIG. 15). Due to the necessary procedure for this staining, cells not oriented in a tubular formation will be washed away. Images of the stained cells were analyzed using Threshold Analysis on MetaMorph®, allowing us to measure the percent of each image covered by endothelial cells and thus tubular structures. The results from the average of 3 images at 63× can be seen in FIG. 16. As can be seen, the addition of the fiber to the Matrigel™ has no effect on tubular formation (FIGS. 16, A and B). The injection of 600 mM S1P into the lumen of the fiber does not induce tubular formation as well as 5 μM S1P in the media (FIGS. 16, C and D), but the injection of 1200 mM and 1800 mM S1P into the lumen of the fiber induces tubular formation more than 5 μM S1P in the media (FIGS. 16, C, E and F).

### Applications

These data show that we have created a system that can be used to test the ability of hollow fibers to deliver small molecules (as well as larger proteins) to an environment in which new blood vessels can form. This system, when applied to tissue engineering of capillaries from HUVECs is able to simulate the natural environment endothelial cells are exposed to in the body. As the delivered amount of growth factor increases, the cellular response also increases. Additionally, this system can be altered so that it can be applied to various cell types as well as the growth factors required for these cell types.

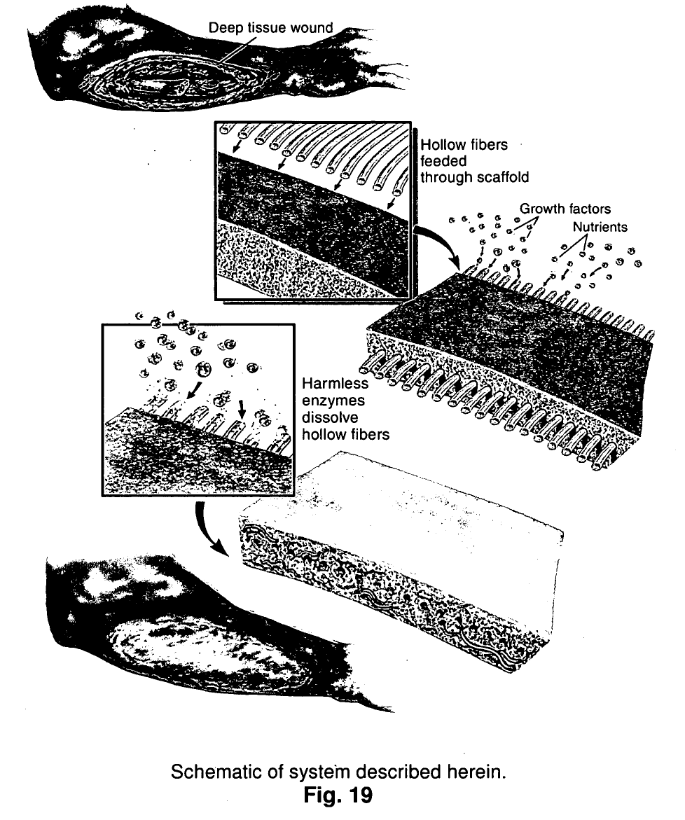

These porous, triggerably-degradable hollow fibers have the potential to be used in parallel to create a larger device that can be used clinically to treat deep wounds (FIG. 19). The choice of growth factor(s) to deliver using this device would depend on the location and severity of the wound. This idea not only allows for targeted delivery of nurtrients and growth factors to wound sites, but also allows for patient-customized treatment.

Additionally, such a device could be used to create three-dimensional tissue in a bioreactor. If a specific tissue is comprised of more than one cell type, the hollow fibers could be arranged in such a fashion that it would deliver specific growth factors to different cell populations. This would also allow for oxygen diffusion beyond the limit of about 1 mm in normal tissue culture, which has been identified as a rate-limiting step in in vitro three dimensional tissue engineering (Malda J, et al., The Roles of Hypoxia in the in vitro Engineering of Tissues. Tissue Eng. 2007 September; 13(9):2153-62).

### EXAMPLE 2

### Dissolvable Hollow Fibers for Delivery in Wound Healing

Inducing angiogenesis has been a major focus of medical and tissue engineering research, as it has the potential to facilitate healing in deep wounds where native vasculature is greatly damaged. Although not presently used for externally regulated release purposes, cellulose hollow fibers (which can be degraded with cellulase) have been previously used as tissue engineering scaffolds. We hypothesize that hollow fibers fabricated from a triggerably-dissolvable material, such as cellulose, can be used to controllably release growth factors to a wound site, while allowing for non-invasive removal. Such a strategy would be externally regulated and also permit true, temporal release. Cellulose hollow fibers were successfully produced using a double injection nozzle in a solvent/non-solvent extraction system. Characterization using Scanning Electron Microscopy (SEM) indicated that fiber porosity can be controlled by adjusting fabrication conditions. The controlled release of fluorescein from these hollow fibers at 37° C. was measured over a nine hour time period. To test our fibers in a system that would simulate a physiological gradient, an angiogenesis promoting factor, sphingosine-1-phosphate (S1P), was released from fibers embedded in Matrigel™ on which human umbilical vascular endothelial cells (HUVECs) were seeded. Tubular formation was observed using phase-contrast microscopy and quantified using histology. Fibers made from cellulose were able to controllably release angiogenic factors in a biologically active form and control the behavior of an endothelial cell population in a dose-specific manner.

### Methods

Hollow Fiber Fabrication. FIG. 6, described above, illustrates how a polymer solution (cellulose) and a miscible antisolvent (water) were extruded through a double injection nozzle into an antisolvent bath for this Example. Beginning in the air gap and continuing into the antisolvent bath, solvent is extracted from the polymer solution, forming a hollow fiber.

In vitro Release Setup. FIG. 1 illustrates the in vitro setup (one well of a six well plate). Two percent agarose gels were cast in 6-well plates, and a 1 cm wide strip was removed from the center axis. This well was filled with the commercially available matrix, Matrigel™ (BD Biosciences®), and a cellulose hollow fiber was embedded before the Matrigel™ set.

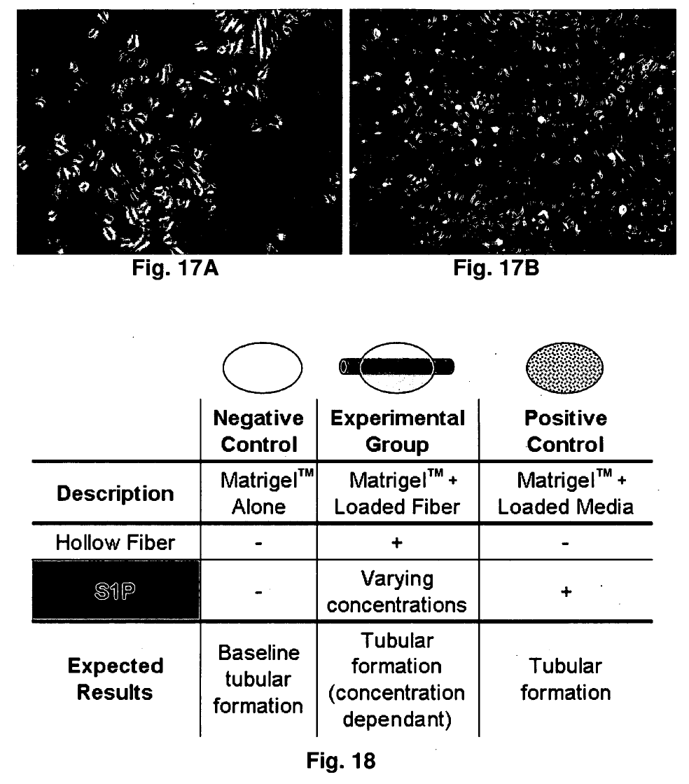

HUVEC Cell Culture. FIGS. 17A-B shows HUVECs (Clonetics®), which were maintained in EBM-2 media (Clonetics® with SingleQuots Kit), 37° C., 5% CO₂. Cells used for experiments were passage 13 of the primary culture. FIG. 17A shows HUVECs at day 3 and FIG. 17B shows HUVECs at confluency.

Experimental Design. FIG. 18 shows a summary of the experimental design. HUVECs were cultured in M199 with 1% FBS for 6 hours. Following trypsinization, cells were pretreated with 100 nM Calphostin C for 30 minutes at room temperature. Cells were then plated on the in vitro setup shown in FIG. 1. Each well was assigned to either the Negative Control, Experimental Group or Positive Control. The Negative Control group consisted of cells cultured on Matrigel™ in M199 with 1% FBS. The Positive Control group consisted of cells cultured on Matrigel™ in M199 with 1% FBS and 5 μM S1P. The Experimental Group consisted of cells cultured on Matrigel™, with embedded hollow fiber, in M199 with 1% FBS. In this group, the hollow fiber was injected with M199 with 1% FBS and 0 mM, 600 mM, 1200 mM and 1800 mM S1P (MW=379).

### Results

Pore morphology. FIG. 8 shows cellulose hollow fibers with varying water flow rates: (A) 10 mL/min, (B) 15 mL/min, and (C) 20 mL/min. An increase in water flow rate increased void fraction. FIG. 9 shows cellulose hollow fibers with varying cellulose flow rates: (A) 1.5 mL/min and B) 2.0 mL/min. An increase in cellulose flow rate increased void fraction.

Fluorescein Release Profile. FIG. 12 shows a graph of a fluorescein release profile, where 50 μL of fluorescein (200 ug/mL, MW=330) was injected into the lumen of the fiber and 1 mL of media (M199) was added on top of the gels. Gels were incubated at 37° C. and fluorescein release was measured by sampling the supernatant and measuring fluorescence emissions over 9 hours. Preliminary results demonstrate that fluorescein can be released from the fiber and through the Matrigel™ for approximately 1.5 hours in a reasonably linear fashion. After this time, the release rate drops and levels off to zero for the remainder of the assay.

Tubular Formation Assay. FIG. 14 shows brightfield tubular formation images of HUVECs plated on Matrigel™, 10×: (A) No fiber, M199, (B) M199 injected into fiber, (C) No fiber, 5 μM S1P in M199, (D) 600 mM S1P injected into fiber, (E) 1200 mM S1P injected into fiber, and (F) 1800 mM S1P injected into fiber. These results demonstrate that hollow fibers embedded in Matrigel™ are able to deliver active S1P to HUVECs. FIG. 15 shows tubular formation image of HUVECs plated on Matrigel™ stained with Rhodamine Phalloidin (for F-actin), 60×. FIG. 16 shows the percent area covered by HUVECs plated on Matrigel™ for HUVECs treated with: (A) No fiber, M199, (B) M199 injected into fiber, (C) No fiber, 5 μM S1P in M199, (D) 600 mM S1P injected into fiber, (E) 600 mM S1P injected into fiber, and (F) 600 mM S1P injected into fiber. This quantitative data demonstrates when S1P is delivered to HUVECs via hollow fibers embedded in Matrigel™, tubular formation occurs in a dose-dependent manner.

Conclusion. Cellulose hollow fibers were produced with varying porosities using a double injection solvent extraction system. Fiber porosity (and corresponding release properties) can be easily controlled by the fabrication conditions. An in vitro HUVEC/Matrigel™ system (FIG. 1) was devised to mimic a growth factor gradient through the extracellular matrix material. Embedded fibers were used to controllably release the angiogenesis promoting molecule S1P to HUVECs seeded on Matrigel™. The extent of tubular formation of these HUVECs was directly proportional to the concentration of S1P loaded into the embedded hollow fiber. This system has potential for facilitating angiogenesis in wounds that cannot heal because the native vasculature is too greatly damaged.

### EXAMPLE 3

### Temporal Delivery of Growth Factors for Wound Healing using Porous Hollow Fibers

One of our objectives is to optimize wound healing through temporal delivery of growth factors using porous hollow fibers extending into a wound site. As an extension to the wound-cap technology (artificial capillary bed delivery system), these fibers can be made from materials that dissolve in the presence of a chemical or temperature-based trigger following the wound healing process. Because angiogenesis is, in many cases, one of the first steps towards wound healing, we propose to demonstrate enablement of this technology by mimicking the natural sequence of stimuli that directs angiogenesis. We believe that sequential delivery of appropriate angiogenesis-promoting factors from our externally-regulated delivery system, as opposed to simultaneous delivery of multiple factors, will result in more mature and integrated neo-vasculature.

Normal wound healing involves the presentation of many growth factors and signals from nearby cells and microvasculature (Bates D O and Jones R O, The role of vascular endothelial growth factor in wound healing. Int J Lower Extremity Wounds, 2003. 2(2): 107-20). In healthy tissue, a significant portion of this task is accomplished through the host's native vasculature. However, in large wounds, the adjacent cells and microvasculature are damaged to the extent that they are incapable of this task, severely limiting proper wound healing (Martin Y and Vermette P, Bioreactors for tissue mass culture: Design, characterization, and recent advances. Biomaterials, 2005. 26:7481-503; Chapekar M S, Tissue engineering: challenges and opportunities. J Biomed Mat Res., 2000. 53(6):617-20). The same issue currently limits in vitro tissue engineering as factor diffusion over distances greater than 1 mm is insufficient for nutrients and growth factors supply (Shimizu T, et al., Polysurgery of cell sheet grafts overcomes diffusion limits to produce thick, vascularized myocardial tissues. FASEB J., 2006. 20(6):708-10). As a non-limiting example, one of our long-term objectives is to develop a therapeutically-relevant artificial wound capillary bed that can provide the appropriate nutrients and growth factors for the corresponding stage of tissue regeneration (FIG. 19). The wound cap technology is based upon the idea that porous, hollow fibers can provide a way to externally control delivery of nutrients and growth factors to a wound site over time. So that these fibers can have eventual therapeutic relevance in deeper wounds, we are currently utilizing materials that are susceptible to chemical or temperature “triggers” (either temperature- or chemical-based) which can be applied to the lumen following wound healing, degrading or dissolving the fibers. One example of such a material is cellulose, a natural biocompatible material that can be degraded by the enzyme cellulase without affecting mammalian tissue (Ko I K and Iwata H. Ann NY Acad Sci, 2001. 944: 443-55).

Inducing angiogenesis is, in many cases, one of the first steps towards in situ wound healing. Researchers have been taking advantage of this by developing therapeutic wound healing strategies that focus on angiogenesis (Tabata Y, et al., Controlled release of vascular endothelial growth factor by use of collagen hydrogels. J Biomat Sci Polym ed., 2000. 11(9): 915-30; Kawanabe T, et al., Sphingosine 1-phosphate accelerates wound healing in diabetic mice. J Dermatol Sci, 2007. 48(1): 53-60). VEGF (45 kD) is a known initiator of angiogenesis (Richardson T P, et al., Polymeric system for dual growth factor delivery. Nat. Biotech., 2001. 19(11): 1029-34), playing a major role in vasculature permeability and endothelial cell recruitment (Bouïs D, et al., A review on pro- and anti-angiogenic factors as targets of clinical intervention. Pharmacol Res., 2006. 53(2): 89-103). However, when delivered as a single factor to ischemic limbs, it has induced the formation of leaky and immature vasculature (Bouïs D, et al., Pharmacol Res., 2006. 53(2): 89-103). Basic FGF (17 kDa) has been known to induce endothelial cell proliferation, and has also been shown to enhance collateral blood flow when administered to an ischemic coronary artery (Unger E F, et al., Basic fibroblast growth factor enhances myocardial collateral flow in a canine model. Am J Physiol, 1994. 266(4 pt 2): H1588-95), limb and heart (Nakajima H, et al., Therapeutic angiogenesis by the controlled release of basic fibroblast growth factor for ischemic limb and heart injury: toward safety and minimal invasiveness. J Artif Organs, 2004. 7(2): 58-61). Additionally, both VEGF and bFGF have been shown to play a major role in the initiation (sprouting) of new capillaries in vivo (Borges J, et al., In vitro analysis of the interactions between preadipocytes and endothelial cells in a 3D fibrin matrix. Minim Invasive Ther Allied Technol., 2007. 16(3): 141-8).

However, it has been shown that bFGF-induced tubular structures will regress over time without the presence of other signals (Simons M, et al., Clinical trials in coronary angiogenesis: issues, problems, consensus: An expert panel summary. Circulation, 2000. 102(11): E73-86). S1P has also been shown to promote vessel stabilization in vivo (Wacker B K, et al., Biomacromolecules, 2006. 7(4): 1335-43; Lee O H, et al., Sphingosine 1-phosphate induces angiogenesis: its angiogenic action and signaling mechanism in human umbilical vein endothelial cells. Biochem Biophys Res Comm., 1999. 264: 743-50). PDGF is released from activated platelets (Wacker B K, et al., Biomacromolecules, 2006. 7(4): 1335-43), and has been shown to promote the maturation of blood vessels through the recruitment and support of mural cells (Richardson T P, et al., Nat. Biotech., 2001. 19(11): 1029-34; Magnusson P U, et al., Platelet-Derived Growth Factor Receptor-{beta} Constitutive Activity Promotes Angiogenesis in vivo and in vitro. Arterioscler Thromb Vasc Biol., 2007. 27(10): 2142-9). In practice, PDGF has been licensed as a treatment for neuropathic diabetic foot ulcers (Bouïs D, et al., Pharmacol Res., 2006. 53(2): 89-103). In light of these results, some investigators have hypothesized that a combination of angiogenic growth factors might be the key to inducing functional angiogenesis that integrates with native vasculature (Richardson T P, et al., Nat. Biotech., 2001. 19(11): 1029-34; Bouïs D, et al., Pharmacol Res., 2006. 53(2): 89-103; Peattie R A, et al., Stimulation of in vivo angiogenesis by cytokine-loaded hyaluronic acid hydrogel implants. Biomaterials, 2004. 25(14): 2789-98; Simmons C A, et al., Dual growth factor delivery and controlled scaffold degradation enhance in vivo bone formation by transplanted bone marrow stromal cells. Bone, 2004. 35(2): 562-9).

Conversely (without wishing to be limited by theory), we have reason to believe that in situ angiogenesis is a complex, multistage, cascade of events guided by a sequence of several growth factors including VEGF, fibroblast growth factor (FGF), PDGF, epidermal growth factor (EGF) as well as coagulation factors, proteinases (Bouïs D, et al., Pharmacol Res., 2006. 53(2): 89-103) and cellular metabolites such as sphingosine-1-phosphate (S1P) (Wacker B K, et al., Biomacromolecules, 2006. 7(4): 1335-43). This implies that presenting more than one factor at the same time may not always be ideal. For instance, it has been shown that when basic FGF (bFGF) and PDGF are presented simultaneously, PDGF seems to inhibit the angiogenic effects of bFGF (De Marchis F, et al., Platelet-derived growth factor inhibits basic fibroblast growth factor angiogenic properties in vitro and in vivo through its alpha receptor. Blood, 2002. 99(6): 2045-53), suggesting that some angiogenic growth factors might be inhibitory at certain stages of angiogenesis. The same conclusion could be made in light of data demonstrating that S1P inhibits recruitment of endothelial cells (Wendler C C and Rivkees S A, Sphingosine-1-phosphate inhibits cell migration and endothelial to mesenchymal cell transformation during cardiac development. Dev Biol., 2006. 291(2): 264-77), a task that is performed by VEGF. Furthermore, VEGF seems to upregulate S1P receptors on endothelial cells (Igarashi J, et al., VEGF induces S1P1 receptors in endothelial cells: Implications for crosstalk between sphingolipid and growth factor receptors. Proc Nat Acad Sci USA, 2003. 100: 10664-9), indicating that VEGF plays a role in angiogenesis before S1P. Collectively, these results suggest that in order to achieve stable, mature vasculature, it may be important to introduce these factors during the appropriate stage of angiogenesis.

With respect to controlled release systems, little has been done to enable technologies capable of a complex, temporal delivery scheme. Recently, dual, controlled delivery of two of the aforementioned factors has also been attempted (VEGF and PDGF) using a combination of multiple delivery systems (Richardson T P, et al., Polymeric system for dual growth factor delivery. Nat. Biotech., 2001. 19(11): 1029-34; Chen R R, et al., Spatio-temporal VEGF and PDGF delivery patterns blood vessel formation and maturation. Pharmaceut Res, 2007. 24(2): 258-64). In this work, the investigators compared bolus delivery of VEGF and PDGF to controlled release of these two factors in vivo. It was found that controlled delivery of both of these factors led to more mature vasculature over a 4 week period than the corresponding bolus injection. They speculated the different release profiles for each factor may have aided in the observed response. Although controlled release of the factors was beneficial, appropriate controls were not present to test the true effect of temporal release (Chen R R, et al., Pharmaceut Res, 2007. 24(2): 258-64; Hao X, et al., Angiogenic effects of sequential release of VEGF-A165 and PDGF-BB with alginate hydrogels after myocardial infarction. Cardiovasc Res., 2007. 75(1): 178-85). It is possible (even likely) that the controlled delivery alone caused the improvement in response. In order to study an optimal delivery strategy for the numerous angiogenic factors, a modular delivery system must be devised to study of any number of factors in sequence. To our knowledge, no one has yet studied the sequential delivery of several regenerative factors in a way that allows for true temporal control over the presence or absence of these factors. However, the wound cap technology (based upon porous, hollow fibers) would enable such a temporal delivery strategy.

We have extended the wound cap technology using materials that would dissolve/degrade following wound healing by using a chemical or temperature trigger. Thus far, we have demonstrated that we have control over the size, thickness, and porosity of cellulose hollow fibers (by varying the fabrication conditions) and therefore can control the rate and duration of release for a broad range of factors. These molecules include sphingosine-1-phospate (S1P, 379 Da), as well as proteins as large as 66 kDa. Furthermore, we have shown that S1P can be delivered to endothelial cells through an extracellular matrix, using these hollow fibers. By varying the concentrations of factor in the lumen of the fiber, we are capable of controlling endothelial cell behavior in a dose-dependent manner. These results have recently been extended to a murine Matrigel™ plug model in which we are capable of delivering a temporal schedule of angiogenesis promoting factors and measure the resulting effect by examining the induction of neovasculature.

Research Design and Methods. Using our modified wound cap fibers, we may characterize temporal delivery in two, recently highlighted sets of angiogenesis promoting factors in vivo. The first strategy intends to deliver basic fibroblast growth factor (bFGF, 17 kDa, early-stage factor) followed by platelet derived growth factor (PDGF, 25 kDa, late-stage factor). The second strategy includes vascular endothelial growth factor (VEGF, 45 kDa) and S1P (379 Da) in which VEGF (early-stage factor) may be delivered before S1P (late-stage factor).

In vivo angiogenesis may be measured using the sponge/Matrigel™ assay (Akhtar N, Dickerson E B, and Auerbach R, The sponge/Matrigel angiogenesis assay. Angiogenesis, 2002. 5: 75-80), a model where a gel implant establishes a cell-free zone from which factors are released for measuring angiogenesis. This cell-free zone ensures that any cells present in the Matrigel™ plug, at the experimental endpoint, are cells that have migrated to the site, as opposed to existing vasculature, which is present in native tissue. Briefly, Matrigel™ may first be injected subcutaneously on the dorsal side of a C57/BL6 mouse. A hypodermic needle may then be inserted into the plug, exiting through the skin. A fiber may be threaded through the needle, which can be subsequently removed, leaving behind a single cellulose hollow fiber embedded in a Matrigel™ plug. This fiber gives us external control of the growth factors that can be delivered to the Matrigel™ plug, developing a gradient.

Both sets of factors described above may be compared using sequential delivery and dual delivery of the same factors. An established method of quantifying new, integrated vasculature may be implemented by injecting FITC-Dextran into the tail vein of a mouse measuring the total fluorescent signal after resecting and dissolving the plug (Akhtar N, et al. Angiogenesis, 2002. 5: 75-8; Chander S K, et al., in vivo inhibition of angiogenesis by sulphamoylated derivatives of 2-methoxyoestradiol. Br J Cancer, 2007. 96(9): 1368-76; Guedez L, et al., Quantitative assessment of angiogenic responses by the directed in vivo angiogenesis assay. Am J Pathol., 2003. 162(5): 1431-9). Further assessment may be performed by histological tissue explant analysis of cells that positively stain for vonWillebrand factor (vWF) and proliferating cell nuclear antigen (PCNA). We may also examine the presence of RGS5 positive cells surrounding neo-vasculature, signifying mature vessels. These studies might allow us to measure: 1) the total amount of angiogenesis, 2) integration of neovasculature with host vasculature, and 3) the maturity of new vessels. We expect that temporal release of these factors according to natural angiogenesis mechanism will result in more mature, integrated neo-vasculature than when the same factors are delivered simultaneously.

Military Relevance. Although battlefield fatality is less than half of what it has been in previous wars, battlefield injury is at its highest (Connolly, C., U.S. Combat Fatality Rate Lowest Ever, in Washington Post, at A24 (Dec. 9, 2004); 29. Vlahos, K. B. Soldiers' Lives Saved But Injuries Persist Long After Battle. Foxnews.com (Nov. 28, 2006)). These wounds are often associated with profuse bleeding, pain, inflammation and infection, leading to increased pressure, reduced blood flow (ischemia) and eventually a condition known as compartment syndrome and/or set the stage for wound regeneration. Proper reestablishment of blood flow in a wound has potential to eliminate compartment syndrome and set the stage for regeneration. Furthermore, the temporal delivery system may also be applied to expedite proper healing in other wound healing milieus such as bone/craniofacial where temporal delivery is critical to simulating the natural wound healing process.

Public Purpose. Diabetic ulcers, caused by poor circulation, afflicts between 600,000 and 800,000 diabetics each year. There are a variety of artificial skin products available to cover a wound that cannot be covered with a patient's own skin; however, these products are limited by the ability of oxygen to diffuse only 1 mm from the surface. A device capable of healing deep wounds has the ability to decrease the amount of trauma related deaths as well as trauma, and diabetic ulcer related amputations and improper healing, while simultaneously reducing health care costs, showing relevance of this work to public health.

Linkage to Previous ARM Studies. A previous ARM study describes the ability of flexible hollow fibers that can continuously perfuse a wound site with a variety of signal molecules and other clinically relevant factors. While that system allows oxygen and nutrients to be delivered to a surface wound, our system can take this technology a step further in order to explore the potential for growth factor delivery in deep wounds. Angiogenic factors can set the stage for wound healing by recruiting the necessary vasculature that can subsequently supply the wound with oxygen and nutrients. This system also utilizes materials that can be triggerably degraded, as our hollow fibers can become embedded in neo-tissue where removal can cause unwanted damage.

### EXAMPLE 4

### In Vivo Murine Matrigel™ Plug Assay for Evaluating Release from Cellulose Hollow Fibers

In this section, we explore the use of porous cellulose hollow fibers in a murine Matrigel™ plug in order to develop a new externally regulated assay system for studying delivery of angiogenic factors. Specific objectives are as follows: 1) fabrication of porous cellulose hollow fibers; 2) in vitro delivery of angiogenic growth factors to control cellular behavior; and 3) in vivo delivery of angiogenic growth factors in a temporal manner.

These objectives were evaluated by examining SEM images of porous cellulose hollow fibers and endothelial cell response to angiogenesis promoting factors both in vitro and in vivo.

Results. The hypothesis driving our work is that temporal growth factor delivery will improve cellular response and eventually wound healing processes. In order to test this hypothesis, we are in need of an assay system capable of detecting changes in angiogenic response while being amenable to sequential delivery schemes. We proposed to utilize porous hollow fibers to deliver angiogenesis promoting factors in an externally regulated manner to endothelial cells in vitro and a subcutaneous Matrigel™ plug in vivo.

Fiber Fabrication. Polymer solution and antisolvent are injected separately into a coaxial nozzle for extrusion. Beginning in the air gap, and continuing into an antisolvent bath, solvent is extracted from the polymer solution, precipitating a porous, hollow fiber. SEM images of porous cellulose hollow fibers are shown in FIGS. 13A-B. A void fraction of 32.2% (determined by Metamorph® threshold image analysis) was obtained when the cellulose solution used to fabricate the hollow fibers contained 1% deionized water, cellulose solution flow rate is 1.5 mL/min and deionized water flow rate is 10 mL/min.

In vitro Tubular Fo nation Assay. Two percent agarose gels were cast in 6-well plates, and a 1 cm wide strip was removed from the center axis. This well was filled with Matrigel™ (BD Biosciences®), and a cellulose hollow fiber was embedded before the Matrigel™ set. This setup was used to evaluate tubular formation and migration of HUVECs when VEGF, PDGF and S1P, are delivered in vitro.

Release of active angiogenesis promoting factors was confirmed when a dosedependent, cellular response to sphingosine-1-phosphate released from a Matrigel™ embedded hollow fiber is observed. FIGS. 14A-F shows brightfield images of HUVECs plated on Matrigel™. These images show that S1P can be delivered through a cellulose hollow fiber embedded and Matrigel™ and control the angiogenic response on HUVECs plated on top of the Matrigel™. The tubular formation when S1P is delivered through the hollow fiber is equivalent or better than when S1P is added directly to the media as shown by threshold image analysis on rhodamine phalloidin stained cells (FIG. 16).

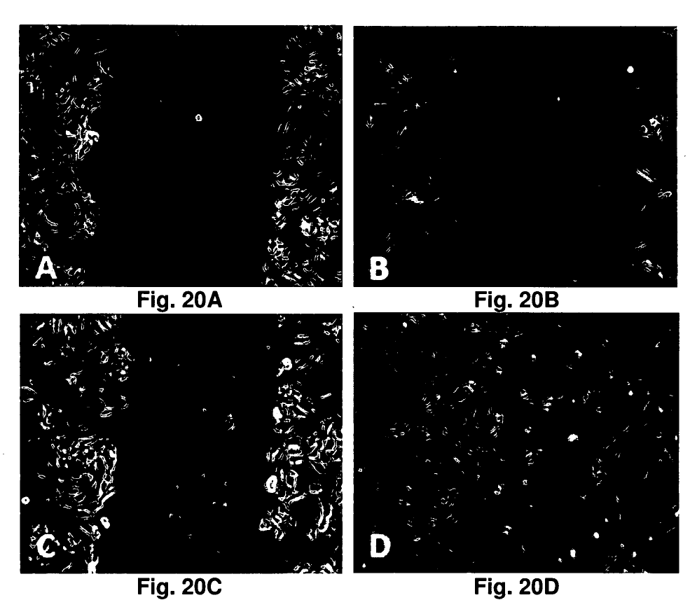

In vitro Scrape Wound Assay. Confluent layers of HUVECs in 6-well plates were scraped with a 100 μL-1000 μL pipette tip and photographed at the time of scrape (FIGS. 20A and 20C). Following the scrape, a cellulose hollow fiber filled with either media (FIG. 20B) or 1800 μM S1P (FIG. 20D) was immersed in the media covering the confluent HUVECs. Cell layers were photographed 42 hours later.

FIG. 20 shows that when S1P was delivered to the media via a hollow fiber, cells increased migration, closing the scrape. This data confirms that when the angiogenesis promoting factor S1P is released through cellulose hollow fibers, they remain active.

In vivo Assay. Growth factor reduced Matrigel™ (500 μL) was injected into the subcutaneous space on the dorsal side of C57BL/6 mice on both the left and right sides. A 12 G needle was used to thread cellulose hollow fibers through the skin and Matrigel™ plugs. Hollow fibers were fixed in place using tissue glue and an Elizabethan collar was used to prevent mice from extracting the hollow fiber. On the day of implantation and every day for the next 6 days, hollow fibers on the left side were injected with sterile saline and hollow fibers on the right side were injected with 10 μL of 100 μg/mL VEGF or 1800 μM S1P (see Table 8 for schedule).

TABLE 8
Schedule of 10 μL injection for right side fiber embedded in subcutaneous Matrigel ™ plug

| | Day 1 | Day 2 | Day 3 | Day 4 | Day 5 | Day 6 | Day 7 |
|---|---|---|---|---|---|---|---|
| VEGF | 100 μg/mL | 100 μg/mL | 100 μg/mL | 100 μg/mL | 100 μg/mL | 100 μg/mL | 100 μg/mL |
| | VEGF | VEGF | VEGF | VEGF | VEGF | VEGF | VEGF |
| S1P | 1800 μM | 1800 μM | 1800 μM | 1800 μM | 1800 μM | 1800 μM | 1800 μM |
| | S1P | S1P | S1P | S1P | S1P | S1P | S1P |
| VEGF followed | 100 μg/mL | 100 μg/mL | 100 μg/mL | 1800 μM | 1800 μM | 1800 μM | 1800 μM |
| by S1P | VEGF | VEGF | VEGF | S1P | S1P | S1P | S1P |
| S1P followed | 1800 μM | 1800 μM | 1800 μM | 100 μg/mL | 100 μg/mL | 100 μg/mL | 100 μg/mL |
| by VEGF | S1P | S1P | S1P | VEGF | VEGF | VEGF | VEGF |

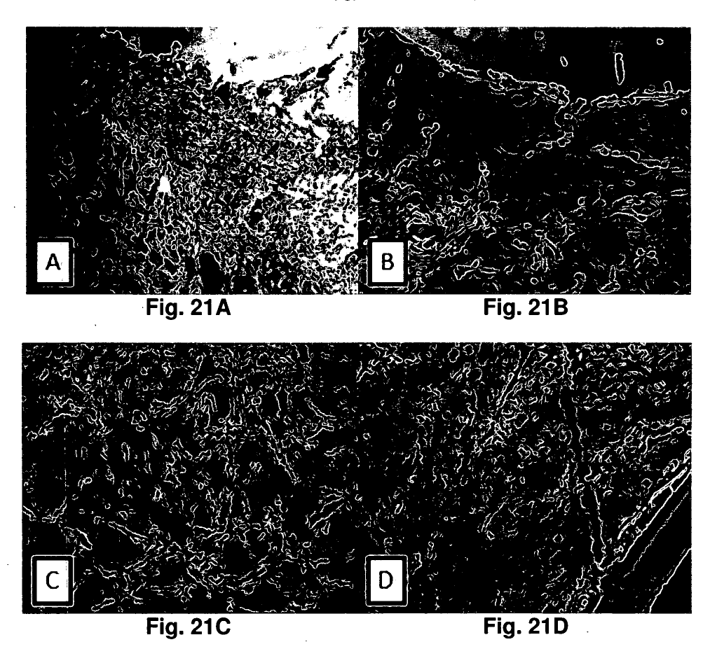

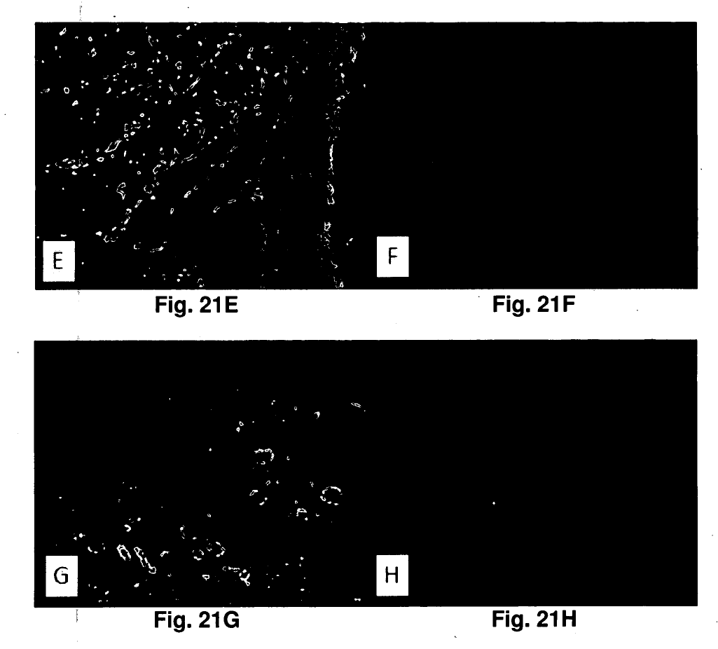

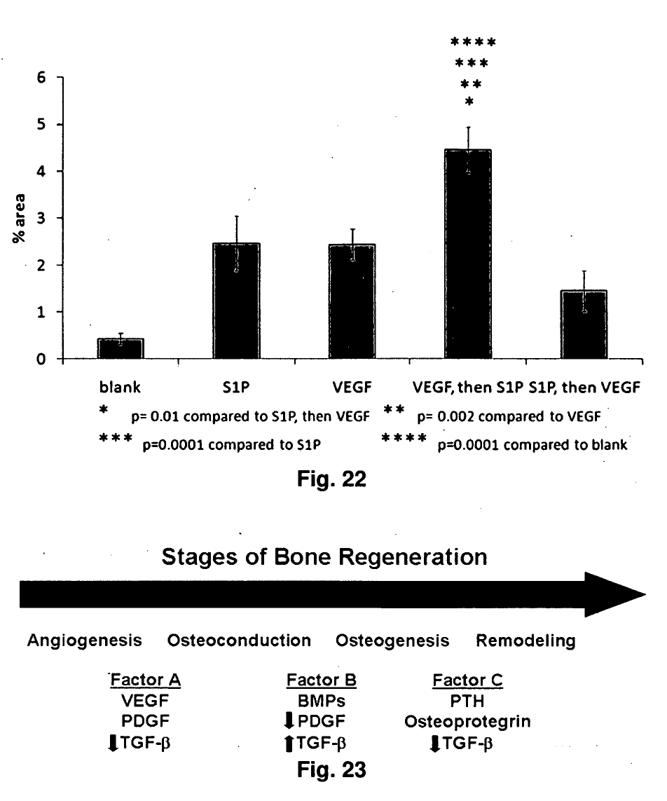

Seven days post-implantation, implants were extracted, fixed and analyzed for endothelial cell migration and vessel formation. H&E images of Matrigel™ plug explants show cellular infiltration in all explanted plugs (FIGS. 21A-D) and blood filled vessels in when VEGF is delivered alone (FIG. 21A) and when VEGF delivery is followed by S1P delivery (FIG. 21C). CD31 staining of Matrigel™ plug explants confirms that cells that have migrated into the Matrigel™ plugs are in fact endothelial cells (FIGS. 21E-H). Metamorph® was used to measure the % area of each image that consisted of endothelial cell staining in order to quantify endothelial cell migration. FIG. 22 shows that the group where VEGF delivery (3 days) was followed by S1P delivery (4 days) has significantly more endothelial cells than any other group (p values determined with ANOVA followed by individual T-tests).

Conclusion. The following are highlights of our accomplishments: 1) ability to alter hollow fiber membrane characteristics based on fabrication procedure; 2) demonstration of the ability to control cellular behavior in a dose-dependent manner when angiogenesis promoting factors are delivered through porous hollow fiber; and 3) temporal delivery of angiogenesis promoting factors in vivo, which reveals that the order in which angiogenesis promoting factors does in fact influence the ability of endothelial cells to migrate into a previously cell-free environment. Not only do these results establish an assay that can be applied to multiple schemas of sequential delivery, but we were also able to use this model to test our original hypothesis involving temporal delivery of growth factors with promising results. We were able to show that the order in which growth factors are delivered for tissue engineering can affect the eventual outcome of the newly forming tissue. These results may have implications for other types of tissue engineering. For example, the many stages of bone tissue engineering (angiogenesis, osteoconduction, osteogenesis, remodeling) coincide with upregulation and down-regulation of specific growth factors (i.e. VEGF, PDGF, TGF-β, BMP). This type of delivery mechanism may be applied to the many stages of wound healing to enhance tissue growth.

### EXAMPLE 5

### Temporal Delivery for Bone

Although battlefield fatality is less than half of what it has been in previous wars, battlefield injuries are at their highest, with most wounds occurring on the head, neck, and limbs. Craniofacial trauma can be particularly devastating to the quality of life of a soldier, interfering with the function of the face and skull thereby affecting the ability to communicate and an individual's self-image. Thus, methods for more efficiently regenerating craniofacial tissue are of utmost interest to our group. Regeneration and de novo formation of bone, like other types tissue regeneration, is a multistage process comprised of differences in stimulus presentation at the various stages. Of particular note, the type of growth factors present at each of the stages of bone regeneration can be distinct, as one would expect to elicit disparate behavior of cells at each stage. Specifically, bone regeneration is commonly divided into four stages: 1) angiogenesis (recruitment of vasculature); 2) osteoconduction (recruitment of osteoprogenitors); 3) osteogenesis (progression of hard tissue formation by osteoprogenitors); and 4) remodeling (hematopoietic progenitors (osteoclasts) become involved to form a structurally sound matrix). The various factors found to be important (or the absence thereof) are shown in FIG. 23. For instance, PDGF is thought to not only promote angiogenesis, but also recruit and differentiate osteoprogenitors (Fiedler J, et al. BMP-2, BMP-4, and PDGF-bb stimulate chemotactic migration of primary human mesenchymal progenitor cells, J Cell Biochem., 2002; 87(3):305-12). However, prolonged exposure to PDGF could suppress mature osteoblast function (Yu X, et al. Temporal expression of PDGF receptors and PDGF regulatory effects on osteoblastic cells in mineralizing cultures, Am J. Physiol., 1997; 272:C1709-16) after progenitor cells colonize the matrix. Conversely, BMPs are commonly known to facilitate osteogenesis (Tsuruga E, et al. Pore Size of Porous Hydroxyapatite as the Cell-Substratum Controls BMP-Induced Osteogenesis. J. Biochem., 1997 February; 121(2):317-24).

In order to study the effects of the temporal presence and absence of these factors, we propose to utilize a porous, hollow fiber delivery system where release can be externally regulated by control over the contents of the fiber lumen at any time. Using this model system, we have demonstrated (in vitro and in vivo) that we are able to control the release of small molecule and protein growth factors by changing the porosity of the fibers while also eliminating the release of a particular factor prior to the onset of release of a second factor. To our knowledge, such precision over the temporal presence and absence of factors has yet to be demonstrated using fully-implantable controlled release vehicles. It is our contention that the hollow fiber system could both: 1) be directly used for clinical growth factor delivery regimens to enhance de novo bone formation in battlefield wounds, and 2) used to understand optimal sequences of delivery that can be programmed into implantable/resorbable systems when appropriate technology becomes available.

We propose to optimize release of two new factors (PDGF, and BMP2) in vitro using established methods and then deliver these factors in sequence in a critically-sized cranial defect in rabbits. This craniofacial model permits placement of the fiber into the critical defect without having to extend through multiple layers of thick, stratified tissue. Following several sequential delivery schemes, micro CT scanning may be performed on live rabbits at various time-points. At the experimental endpoint, tissue sections may be resected and processed via histology and histomorphometry. Negative controls may include animals where hollow fibers are present and deliver saline solution only. We may also perform controls where factors are delivered simultaneously (shown previously to slow bone growth) and also in the opposite temporal sequence (in order to establish that the order of factor delivery is critical). The most effective delivery scheme may be identified using measurements total area of mineralized tissue (microCT) and histological analysis to identify new bone growth via the presence of DMP-1 and bone sialoprotein-positive tissue.

We claim:

1. A tubular structure comprising hollow fibers of a biocompatible, triggerably-dissolvable material.

2. The tubular structure of claim 1, wherein the biocompatible, triggerably-dissolvable material comprises a water-insoluble polysaccharide.

3. The tubular structure of claim 2, wherein the biocompatible, triggerably-dissolvable material comprises a cellulose.

4. The tubular structure of claim 3, wherein the cellulose is cellulose acetate.

5. The tubular structure of claim 1, wherein the material has a void fraction of more than about 0.10.

6. The tubular structure of claim 5, wherein the material has a void fraction of more than about 0.25.

7. The tubular structure of claim 1, wherein the biocompatible, triggerably-dissolvable material comprises a copolymer.

8. The tubular structure of claim 7, in which the copolymer has a lower critical solution temperature of less than 30° C.

9. The tubular structure of claim 7, in which the copolymer comprises an N-alkyl acrylamide residue in which the alkyl is one of methyl, ethyl, propyl, isopropyl and cyclopropyl; one or both of acrylic acid and methacrylic acid; and an acrylic residue having an amine-reactive group, the copolymer comprising a polyester linkage in its backbone.

10. The tubular structure of claim 9, in which the N-alkyl acrylamide is N-isopropylacrylamide

11. The tubular structure of claim 9, wherein the amine-reactive group is one of a succinimide group, an oxysuccinimide group and an isocyanate group.

12. The tubular structure of claim 9, comprising an acrylic acid residue.

13. The tubular structure of claim 9, wherein the copolymer has a lower critical solution temperature above 37° C. after its ester bonds are hydrolyzed.

14. The tubular structure of claim 9, wherein the backbone of the copolymer comprises from 1% to 10% ester bonds.

15. The tubular structure of claim 9, wherein the backbone of the copolymer comprises a polyester macromer.

16. The tubular structure of claim 15, wherein the polyester macromer comprises hydroxyethyl methacrylate and lactide residues.

17. The tubular structure of claim 16, wherein the ratio of hydroxyethyl methacrylate to lactide residues in the polyester macromer ranges from 1:1 to 1:10.

18. The tubular structure of claim 16, wherein the ratio of hydroxyethyl methacrylate to lactide residues in the polyester macromer ranges from 1:2 to 1:8.

19. The tubular structure of claim 15, wherein the polyester macromer comprises hydroxyethyl methacrylate and trimethyl carbonate residues.

20. The tubular structure of claim 19, wherein the ratio of hydroxyethyl methacrylate to trimethyl carbonate residues in the polyester macromer ranges from 1:1 to 1:10.

21. The tubular structure of claim 19, wherein the ratio of hydroxyethyl methacrylate and trimethyl carbonate residues in the polyester macromer ranges from 1:2 to 1:5.

22. The tubular structure of claim 9, comprising one or more of a caprolactone, a glycolide and a trimethylene carbonate residue.

23. The tubular structure of claim 9, further comprising an amine-containing compound attached to the copolymer.

24. The tubular structure of claim 23, wherein the amine-containing compound is collagen.

25. The tubular structure of claim 24, comprising between 1% wt and 10% wt collagen.

26. The tubular structure of claim 23, wherein the amine-containing compound is gelatin.

27. The tubular structure of claim 1, in which the material has an elastic modulus of from 0.5 MPa to 1 MPa.

28. The tubular structure of claim 1, in which a portion of the hollow fibers is embedded in a cell growth scaffold.

29. The tubular structure of claim 28, in which the scaffold comprises extracellular matrix-derived material.

30. The tubular structure of claim 29, wherein the extracellular matrix-derived material is a hydrogel.

31. The tubular structure of claim 29, wherein the extracellular matrix-derived material is Matrigel™.

32. The tubular structure of claim 29, wherein the extracellular matrix-derived material is a collagen.

33. The tubular structure of claim 28, in which the scaffold comprises a copolymer.

34. The tubular structure of claim 33, wherein the copolymer is one of:
P(NIPAAm-co-AAc-co-NHS-co-HEMAPLA2.1) 85/6/5/4;
P(NIPAAm-co-AAc-co-NHS-co-HEMAPLA3.9) 85/6/5/4;
P(NIPAAm-co-AAc-co-NHS-co-HEMAPLA7.0) 85/6/5/4;
P(NIPAAm-co-AAc-co-NHS-co-HEMAPLA2.1) 80/6/5/9;
P(NIPAAm-co-AAc-co-NHS-co-HEMAPLA3.9) 80/6/5/9;
P(NIPAAm-co-AAc-co-NHS-co-HEMAPLA2.1) 75/6/5/14;
P(NIPAAm-co-AAc-co-NHS-co-HEMAPLA3.9) 75/6/5/14;
P(NIPAAm-co-AAc-co-NHS-co-HEMAPLA2.1) 85/6/4/4
P(NIPAAm-co-NHS-co-AAc) 90/0/10;
P(NIPAAm-co-NHS-co-AAc) 90/10/0;
P(NIPAAm-co-NHS-co-AAc) 93.5/0/6.5; and
P(NIPAAm-co-NHS-co-AAc) 93.5/6.5/0.

35. A method of delivering a composition to a patient, comprising:
(a) implanting within the patient a cell growth scaffold comprising a plurality of hollow fibers of a biocompatible, triggerably-dissolvable material, wherein the hollow fiber comprise a first portion embedded in the cell growth scaffold and a second portion extending from the cell growth scaffold; and
(b) injecting the composition into the hollow fibers.

36. The method of claim 35, wherein the biocompatible, triggerably-dissolvable material comprises a cellulose.

37. The method of claim 35, wherein the biocompatible, triggerably-dissolvable material comprises a copolymer having a lower critical solution temperature of less than 30° C.

38. The method of claim 37, in which the copolymer comprises an N-alkyl acrylamide residue in which the alkyl is one of methyl, ethyl, propyl, isopropyl and cyclopropyl; one or both of acrylic acid and methacrylic acid; and an acrylic residue having an amine-reactive group, the copolymer comprising a polyester linkage in its backbone.

39. The method of claim 38, in which the copolymer comprises a polyester macromer.

40. The method of claim 39, in which the polyester macromer comprises hydroxyethyl methacrylate and lactide residues.

41. The method of claim 37, wherein the copolymer is one of:
P(NIPAAm-co-AAc-co-NHS-co-HEMAPLA2.1) 85/6/5/4;
P(NIPAAm-co-AAc-co-NHS-co-HEMAPLA3.9) 85/6/5/4;
P(NIPAAm-co-AAc-co-NHS-co-HEMAPLA7.0) 85/6/5/4;
P(NIPAAm-co-AAc-co-NHS-co-HEMAPLA2.1) 80/6/5/9;
P(NIPAAm-co-AAc-co-NHS-co-HEMAPLA3.9) 80/6/5/9;
P(NIPAAm-co-AAc-co-NHS-co-HEMAPLA2.1) 75/6/5/14;
P(NIPAAm-co-AAc-co-NHS-co-HEMAPLA3.9) 75/6/5/14;
P(NIPAAm-co-AAc-co-NHS-co-HEMAPLA2.1) 85/6/4/4
P(NIPAAm-co-NHS-co-AAc) 90/0/10;
P(NIPAAm-co-NHS-co-AAc) 90/10/0;
P(NIPAAm-co-NHS-co-AAc) 93.5/0/6.5; and
P(NIPAAm-co-NHS-co-AAc) 93.5/6.5/0.

42. The method of claim 35, in which the scaffold comprises an extracellular matrix-derived material.

43. The method of claim 42, wherein the extracellular matrix-derived material is Matrigel™.

44. The method of claim 35, further comprising solubilizing or eroding the biocompatible, triggerably-dissolvable material after injecting the composition into the hollow fibers.

45. The method of claim 35, in which the composition comprises one or more of VEGF, PDGF, S1P, bFGF, and a therapeutic agent.

46. The method of claim 35, wherein the hollow fibers comprise a third portion that extends from the scaffold that is opposite the second portion.

47. A cell culture apparatus comprising a culture vessel containing the tubular structure of claim 1, the tubular structure either extending outside the culture vessel or being connected to a fluid connector that extends a fluid path from the tubular structure to outside the culture vessel.

48. The cell culture apparatus of claim 47, comprising a fluid connector fluidly connected to the tubular structure that extends a fluid path from the tubular structure to outside the culture vessel, the fluid connector comprising a valve.

49. The cell culture apparatus of claim 47, further comprising a cell growth scaffold in which the tubular structure is embedded within the culture vessel.

50. A method of culturing cells or tissue, comprising culturing cells in the cell culture apparatus of claim 47.

51. A method of growing tissue, comprising in a cell culture apparatus or in vivo, contacting cells with a cell growth scaffold comprising a plurality of hollow fibers of a biocompatible, triggerably-dissolvable material comprising a first portion embedded in the cell growth scaffold and a second portion extending from the cell growth scaffold; and administering one or more of a cell growth nutrient, a growth factor and a therapeutic agent to the scaffold through the tubular structure.

52. The method of claim 51, further comprising dissolving all or part of the tubular structure after administering the one or more of a cell growth nutrient, a growth factor and a therapeutic agent to the scaffold through the tubular structure.
